(19) United States
(12) Patent Application Publication
(10) Pub. No.: US 2025/0375122 A1
(43) Pub. Date: Dec. 11, 2025

(54) METHOD AND SYSTEM FOR MANAGING MEDICAL DEVICE WIRELESS DATA TRANSMISSION WITH PACKET FUNCTIONAL INCORPORATION

(71) Applicant: Pacesetter, Inc., Sylmar, CA (US)

(72) Inventor: Xing Pei, Ventura, CA (US)

(21) Appl. No.: 19/092,021

(22) Filed: Mar. 27, 2025

Related U.S. Application Data
(60) Provisional application No. 63/658,628, filed on Jun. 11, 2024.

(51) Int. Cl. A61B 5/07; A61B 5/00; A61B 5/145; A61M 5/142; H04L 1/00; H04W 4/80

(52) U.S. Cl. CPC A61B 5/076; A61B 5/686; H04L 1/0061; A61B 5/14503; A61M 5/14276; H04W 4/80

(57) ABSTRACT

A system and method are provided for communication between first and second devices utilizing a predetermined protocol. The system and method utilize at least one of communication circuitry or a processor, within one of the first and second devices, for, receiving a data packet; determining whether the data packet exhibits an error; when the data packet exhibits the error, incorporating the data packet into a candidate packet register (CPR) within the one of the first medical device and the second device; analyzing a content the CPR for errors; and based on the analyzing, designating the content of the CPR to be a resultant packet and output the resultant packet as a corrected version of the data packet.

### CROSS-REFERENCE TO RELATED APPLICATIONS

This application claims priority to U.S. Application No. 63/658,628, filed 11 Jun. 2024, the subject matter of which is incorporated herein by reference in its entirety.

### BACKGROUND

The modern medical devices have advanced to use common wireless communication technology for short-range communication (e.g., implantable devices may communicate with another device separated by a few meters), such as low energy Bluetooth (BLE) technology where the medical device operates as the slave and the external device operates as the master in terms of the communication protocol.

Low energy Bluetooth (BLE) technology is a common wireless communication technology that is widely used in headphones, earphones, sensors, heart rate monitors, fitness devices, etc., with mobile operating systems including IOS, Android, etc. The BLE enabled devices share the same frequency spectrum range as classic Bluetooth enabled devices. For example, the BLE protocol defines use of 40 channels, with 2 MHz channel spacing while 37 of the channels are data channels and 3 of the channels are advertising channels. The channels are potentially shared by any BLE enabled devices within range of one another.

It can be imagined that more and more wireless devices will be configured to utilize BLE technology. As the number of BLE enabled devices increases, there will be an increased probability that any one communication link will experience interference, interruptions, and be otherwise hindered due to noise within the BLE defined communication channels. Therefore, it is predicted that there will be increased challenges to ensure real time communication in the presence of many BLE systems.

Various types of medical devices are utilized today including both implanted medical devices and non-implanted but otherwise portable wearable medical devices. Implanted and non-implanted medical devices can also be segmented into devices that delivery a therapy and devices that do not delivery a therapy but instead monitor one or more physiologic characteristics of interest. Examples of therapy delivery devices include implantable cardioverter-defibrillator (ICD) devices, cardiac resynchronization therapy (CRT) devices, pacemakers, subcutaneous ICD devices, non-vascular ICD devices, neurostimulation devices, cochlear devices, hearing-aid devices, drug delivery pumps and the like. Examples of non-therapy monitoring devices include implantable cardiac monitors, non-implantable cardiac monitors, patch-type analyte monitors (e.g., glucose monitors), photoplethsmography (PPG) monitors and the like.

The implantable devices have additional challenges, not necessarily experienced by non-implantable devices, as body tissue attenuates wireless signals much more than a degree of signal attenuation experienced in the air. For example, a commonly accepted good BLE communication strength is approximately −55 dBm to −75 dBm. However, an implantable device located subcutaneously or under the rib cage often needs to operate at a communication strength of approximately −70 dBm to −100 dBm. Further, implantable devices located deeper below the skin surface such as within a chamber of the heart (e.g. a leadless implantable pacemaker) may need to operate at even greater communication strength. Although communications technology has advanced to improve receive sensitivity (e.g., with additional front-end amplifiers), communication challenge remains.

In addition, there is limited opportunity to increase the transmission output power. The transmission output power (TX) is limited by at least two factors in medical devices, 1) the power consumption of the device and 2) the international geographic dependent output power limit regulation. Unlike the implantable medical devices, some non-medical devices that utilize BLE communication, do not require a true real time communication. Hence, in many non-medical applications, the devices can tolerate longer delay in communication of data transfer. The data can also be buffered to ensure the continuous data communication. Even in the cardiac implantable device system, during non-critical task execution, the needs for true real-time is not high, such as during reading of diagnostics, real-time EGM streaming during a session with buffering, etc.

However, for cardiac implantable device systems, during in-clinic tasks, such as in-clinic tests, the demand for true real-time communication without interruption is higher. A communication interrupt could delay the completion of a test or interrupt the test, and therefore it is desirable that for system to minimize re-do tests and limit delays in testing, which limits the number of transmissions retries. It is particularly true when performing induction tests for ventricular arrhythmia episodes and therapy evaluation; the demands for true real-time communication without interruption is high. Although the modern implantable cardiac defibrillator has mitigation for communication loss during an induction test, it is still an unpleasant experience if communication is lost during a ventricular arrhythmia episode; the ventricular arrhythmia episode cannot be monitored on the programmer and the evaluation may be automatically cancelled. In a less critical test operation, such as s post implant R wave test, a communication interruption will interrupt the R wave test and prolong the implant procedure, an unpleasant experience for the implanting physician.

The BLE communication protocol allows/will retry the transmission when transmitted data has an error. BLE communication is segmented in connection interval in which multiple packages could be transmitted. In the BLE standard it states, “Two consecutive packets received with an invalid CRC match within a connection event shall close the event.” This means that when experiencing the error condition, one connection interval allows a maximum of two consecutive error packets. Between each scheduled data transmission, multiple connection intervals can occur allowing multiple retries to increase the successful rate of data transmission, if the total data throughput allows, or the data will be dropped.

### SUMMARY

In accordance with an embodiment, a method is provided for communication between a first medical device and a second device utilizing a predetermined protocol, the method comprising: utilizing at least one of communication circuitry or a processor, within one of the first medical device and the second device, for, receiving a data packet; determining whether the data packet exhibits an error; when the data packet exhibits the error, incorporating the data packet into a candidate packet register (CPR) within the one of the first medical device and the second device; analyzing a content the CPR for errors (also referred to as a CPR error); and based on the analyzing, designating the content of the CPR to be a resultant packet and outputting the resultant packet as a corrected version of the data packet.

Additionally or alternatively, the data packet includes a payload block and an error detection block, the incorporating operation adding the payload and error detection blocks to corresponding payload and error detection blocks of the CPR. Additionally or alternatively, the method further comprises rounding the payload and error detection blocks of the CPR on a bit-by-bit basis, based on a number of data packets added to the CPR. Additionally or alternatively, the analyzing operation includes calculating an error detection value associated with the content of the payload block of the CPR and determining when the error detection value matches the error detection block within the CPR. Additionally or alternatively, the receiving includes receiving an original data packet and a corresponding retry data packet, the incorporating operation including the original data packet in the CPR and adding the retry data packet to the CPR.

Additionally or alternatively, the first medical device (MD) represents at least one of an implantable medical device, an analyte sensor or a drug delivery pump, and the second device represents an external device (ED), wherein the protocol supports transmission of retry data packets, the method comprising: establishing a communications link between the first MD and the ED, the communications link having connection intervals in accordance with the protocol; and wherein the CPR represents a dynamic average check (DAC) packet.

Additionally or alternatively, the receiving, determining, incorporating and analyzing operations are performed a first iteration for an original data packet and are repeated, at least a second iteration, for a first retry data packet presenting an attempt to retransmit the original data packet. Additionally or alternatively, during the first iteration, the CPR is initialized with a payload block and an error detection block from the original data packet, and, during the second iteration, the CPR is updated by adding payload and error detection blocks from the first retry data packet to the payload and error detection blocks in the CPR. Additionally or alternatively, when the first retry data packet is determined to exhibit the error, the method repeating the receiving and determining a third iteration in connection with a second retry data packet representing a second attempt to retransmit the new data packet. Additionally or alternatively, the receiving the data packet includes receiving a set of at least two data packets that, when transmitted, included a same payload block and a same error detection block. Additionally or alternatively, the receiving and determining are repeated in connection with a new data packet, followed by corresponding first and second retry data packets, the new, first retry and second retry data packets exhibiting the error, the incorporating including combining the new, first retry and second retry data packets to form the DAC packet, the DAC packet designated to be error free and output. Additionally or alternatively, prior to the incorporating, the CPR already includes a first payload block and first error detection block, the incorporating operation including incorporating a second data packet into the CPR by applying at least one of a mathematical or logical combination to i) second payload and error detection blocks of the second data packet and ii) the first payload and error detection blocks, respectively.

Additionally or alternatively, the determining whether the data packet exhibits an error applies and the analyzing the content the CPR for errors apply a common error detection algorithm to payload and error detection blocks in the data packet and to payload and error detection blocks in the CPR. Additionally or alternatively, the incorporating operation effectively removed one or more errors from the content of the CPR.

In accordance with embodiments herein a system is provided that comprises: first and second medical devices configured to communicate with one another utilizing a predetermined protocol; and at least one of communication circuitry or a processor, within one of the first medical device and the second device, configured to: receive a data packet; determine whether the data packet exhibits an error; when the data packet exhibits the error, incorporate the data packet into a candidate packet register (CPR) within the one of the first medical device and the second device; analyze a content the CPR for errors; and based on the analysis, designate the content of the CPR to be a resultant packet and output the resultant packet as a corrected version of the data packet.

Additionally or alternatively, the data packet includes a payload block and an error detection block, the incorporate operation adding the payload and error detection blocks to corresponding payload and error detection blocks of the CPR. Additionally or alternatively, the at least one of communication circuitry or processor configured to round the payload and error detection blocks of the CPR on a bit-by-bit basis, based on a number of data packets added to the CPR. Additionally or alternatively, the analyze operation includes calculating an error detection value associated with the content of the payload block of the CPR and determining when the error detection value matches the error detection block within the CPR. Additionally or alternatively, the receive operation includes receiving an original data packet and a corresponding retry data packet, the incorporating operation including the original data packet in the CPR and adding the retry data packet to the CPR. Additionally or alternatively, the first medical device (MD) represents at least one of an implantable medical device, an analyte sensor or a drug delivery pump, and the second device represents an external device (ED), wherein the protocol supports transmission of retry data packets, the first MD and the ED configured to establish a communications link having connection intervals in accordance with the protocol; and wherein the CPR represents a dynamic average check (DAC) packet.

Additionally or alternatively, the receive, determine, incorporate and analyze operations are performed a first iteration for an original data packet and are repeated, at least a second iteration, for a first retry data packet presenting an attempt to retransmit the original data packet. Additionally or alternatively, during the first iteration, the CPR is initialized with a payload block and an error detection block from the original data packet, and, during the second iteration, the CPR is updated by adding payload and error detection blocks from the first retry data packet to the payload and error detection blocks in the CPR. Additionally or alternatively, when the first retry data packet is determined to exhibit the error, the system repeating the receiving and determining a third iteration in connection with a second retry data packet representing a second attempt to retransmit the new data packet. Additionally or alternatively, the receiving the data packet includes receiving a set of at least two data packets that, when transmitted, included a same payload block and a same error detection block.

Additionally or alternatively, the receiving and determining are repeated in connection with a new data packet, followed by corresponding first and second retry data packets, the new, first retry and second retry data packets exhibiting the error, the incorporating including combining the new, first retry and second retry data packets to form the DAC packet, the DAC packet designated to be error free and output. Additionally or alternatively, prior to the incorporating, the CPR already includes a first payload block and first error detection block, the incorporating operation including incorporating a second data packet into the CPR by applying at least one of a mathematical or logical combination to i) second payload and error detection blocks of the second data packet and ii) the first payload and error detection blocks, respectively. Additionally or alternatively, the determining whether the data packet exhibits an error applies and the analyzing the content the CPR for errors apply a common error detection algorithm to payload and error detection blocks in the data packet and to payload and error detection blocks in the CPR. Additionally or alternatively, the incorporating operation effectively removed one or more errors from the content of the CPR.

In accordance with embodiments herein, a method is provided for communication between a first device and a second device utilizing a predetermined protocol. The method utilizes at least one of communication circuitry or a processor, within one of the first device and the second device, for, receiving a data packet and determining whether the data packet exhibits an error. When the data packet exhibits the error, the method incorporates the data packet into a candidate packet register (CPR) within the one of the first device and the second device, analyzes a content the CPR for errors; and based on the analysis, designates the content of the CPR to be a resultant packet and output the resultant packet as a corrected version of the data packet.

Additionally or alternatively, the data packet includes a payload block and an error detection block, the incorporating operation comprises adding the payload and error detection blocks to corresponding payload and error detection blocks of the CPR. Additionally or alternatively, the method further comprises rounding the payload and error detection blocks of the CPR on a bit-by-bit basis, based on a number of data packets added to the CPR. Additionally or alternatively, the analyzing operation includes calculating an error detection value associated with the content of the payload block of the CPR and determining when the error detection value matches the error detection block within the CPR.

Additionally or alternatively, a system is provided that comprises first and second devices configured to communicate with one another utilizing a predetermined protocol. One of the first and second devices includes at least one of communication circuitry or a processor, configured to: receive a data packet and determine whether the data packet exhibits an error. When the data packet exhibits the error, the data packet is incorporated into a candidate packet register (CPR) within the one of the first device and the second device, a content of the CPR is analyzed for errors. Based on the analysis, the content of the CPR is designated to be a resultant packet and the resultant packet is output as a corrected version of the data packet.

Additionally or alternatively, the data packet includes a payload block and an error detection block, the incorporate operation adding the payload and error detection blocks to corresponding payload and error detection blocks of the CPR. Additionally or alternatively, the at least one of communication circuitry or processor is configured to round the payload and error detection blocks of the CPR on a bit-by-bit basis, based on a number of data packets added to the CPR. Additionally or alternatively, the analyze operation includes calculating an error detection value associated with the content of the payload block of the CPR and determining when the error detection value matches the error detection block within the CPR.

### BRIEF DESCRIPTION OF THE DRAWINGS

FIG. 1 illustrates a collection of graphs representing simulations for a relation between the bit error rate and the transmitted packet error rate for a transmitted data packet.

FIG. 2 illustrates a collection of graphs representing simulations for a relation between the bit error rate and the transmitted packet error rate for a transmitted data packet (T1), re-transmitted (retried) packets (R3, R5, R6) and the packet error rate when applying the present processes to form an error corrected resultant data packet (DA3, DA5).

FIG. 3A illustrates examples of environments in which embodiments may be implemented.

FIG. 3B illustrates examples of environments in which embodiments may be implemented.

FIG. 3C illustrates examples of environments in which embodiments may be implemented.

FIG. 4A illustrates a block diagram of communication circuitry 400 utilized in accordance with an embodiment herein.

FIG. 4B illustrates an example protocol stack structure of a Bluetooth protocol.

FIG. 5 illustrates a distributed processing system in accordance with one embodiment.

FIG. 6 illustrates a method for managing communication between at least first and second devices in accordance with embodiments herein.

FIG. 7 illustrates a method for managing communication between at least first and second devices in accordance with embodiments herein.

FIG. 8 illustrates a method for managing communication between at least first and second devices in accordance with embodiments herein.

FIG. 9 illustrates a method implemented in accordance with embodiments herein.

FIG. 10A illustrates an example for managing content of the candidate packet register in connection with an original receive and first and second retries in accordance with an embodiment herein.

FIG. 10B illustrates an example for managing content of the candidate packet register in connection with the original receive and first and second retries of FIG. 10A, followed by third and fourth retries, in accordance with an embodiment herein.

### DETAILED DESCRIPTION

It will be readily understood that the components of the embodiments as generally described and illustrated in the Figures herein, may be arranged and designed in a wide variety of different configurations in addition to the described example embodiments. Thus, the following more detailed description of the example embodiments, as represented in the Figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, etc. In other instances, well-known structures, materials, or operations are not shown or described in detail to avoid obfuscation. The following description is intended only by way of example, and simply illustrates certain example embodiments.

The methods described herein may employ structures or aspects of various embodiments (e.g., systems and/or methods) discussed herein. In various embodiments, certain operations may be omitted or added, certain operations may be combined, certain operations may be performed simultaneously, certain operations may be performed concurrently, certain operations may be split into multiple operations, certain operations may be performed in a different order, or certain operations or series of operations may be re-performed in an iterative fashion. It should be noted that other methods may be used, in accordance with an embodiment herein. Further, wherein indicated, the methods may be fully or partially implemented by one or more processors of one or more devices or systems. While the operations of some methods may be described as performed by the processor(s) of one device, additionally, some or all of such operations may be performed by the processor(s) of another device described herein.

Each and every patent, published patent application, specification and publication referenced herein is expressly incorporated by reference in its entirety.

### A. Base Functioning of Computer and/or Communication Technology

Embodiments herein are described in connection with one or more communications technologies that support real time transmission of data streams, where a corresponding communication protocol segments the data stream into data packets and supports re-transmission of data packets, from a transmitting device, when the receiving device determines that a received data packet includes errors as received. The data packets include, among other things, a header block, a payload block, an error detection block and (optionally) error correction. The computer and/or communications technology provides for data packet transmission during connection events, with one or more data packets transmitted during each connection event. The connection interval is the amount of time between two successive connection events. For example, in the Bluetooth communications protocol, the connection interval may be between 5 ms and 5 seconds and more specifically between 7.5 ms and 4 s.

For example, the BLE protocol defines connection intervals in units that are multiple of 1.25 ms in the range of 7.5 ms to 4.0 s. The BLE protocol allows multiple data packets to be successively transmitted in one connection interval. By way of example, an ICM may transmit up to 6 data packets within one connection interval of 7.5 ms. During real-time data transmission, an ICM transmits segments of EGM data “strips” that have a predetermined length. The length of the EGM data strip may be defined by a clinician based on the type of events of interest. For example, an ICM may store and transmit, to an external device, EGM data strips having a length of approximately 45-50 ms.

The present example assumes that each data packet transmitted is received error free at the external device. However, in many instances, at least a portion of the data packets may exhibit errors and warrant retransmission. The BLE protocol (as well as other protocols) is designed to allow for a predetermined number of data packets to be retransmitted before a current connection event is terminated. For example, when a first data packet transmitted exhibits errors upon receipt, the data packet may be retransmitted multiple times (e.g., up to 11) until the received data packet is error free.

Further, in some implementations (e.g., during clinical visits or home-based tests), it is not practical for the ICM to continuously retransmit the same data packet until it is received error free. For example, during a clinical visit, it is desirable to transmit an EGM data strip in real time. It is important that the data values for the EGM data strip are transmitted as near as practical to the point in time as when measures to maintain the real time nature of the test. Hence, an ICM may be configured to allow only a predetermined number of retransmissions of data packets before moving on and ignoring the erroneous data packets.

In BLE communication, there are two equivalent ways to describe the error rate: 1) the BER (Bit error rate) and the PER (Packet error rate). The PER is a function of the BER and the total number of bits in each data packet. When communication noise increases or when signal strength weakens, the bit error rate and the packet error rate both will increase.

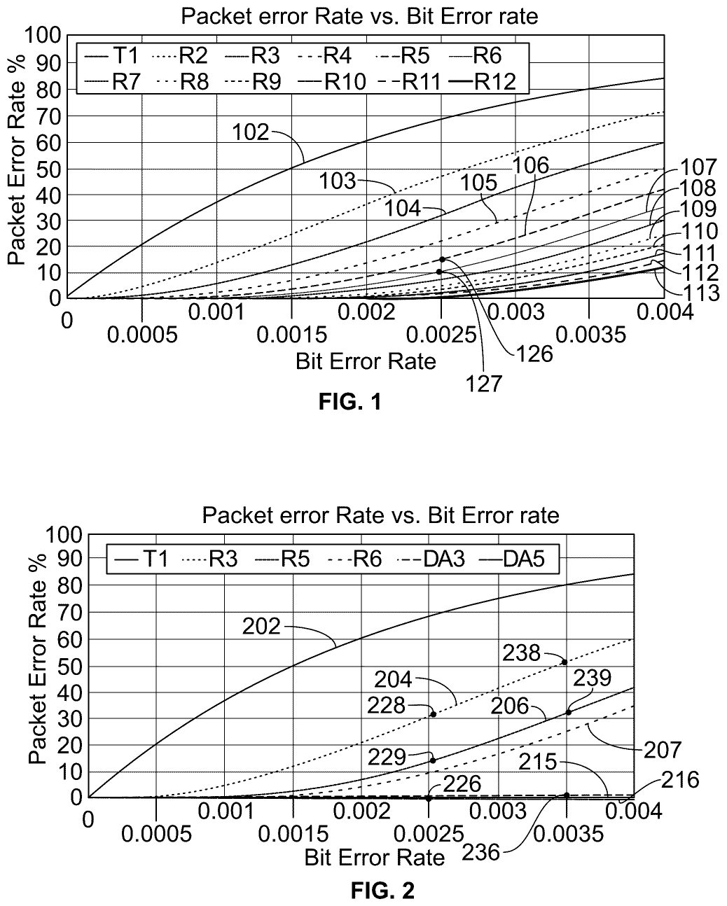

FIG. 1 illustrates a collection of graphs representing simulations for a relation between the bit error rate and the transmitted packet error rate for a transmitted data packet. The horizontal axis denotes a bit error rate that may be experienced by a communications environment. The bit error rate is affected by various factors, such as noise, transmission medium, transmission distance, transmit power levels, receiver sensitivity, wireless technology utilized to transmit and receive data packets, and the like. The vertical axis denotes a rate at which data packets conveyed through the communications environment exhibit errors upon receipt at a receiving device. The simulations of FIG. 1 assume transmission of data packets having 48 bytes of payload. Graphs 102-113 individually correspond to the number of times that a common data packet is retransmitted. Graph 102 corresponds to a new or original data packet (T1) transmitted for the first time. Graph 103 Corresponds to a data packet (R2) that was previously transmitted once, upon arrival was found to exhibit an error and then was retransmitted once (herein referred to as the first retransmission or first retry). Graph 104 corresponds to a data packet (R3) that was previously transmitted twice, upon both prior arrivals was found to exhibit an error and then was retransmitted a second time (herein referred to as the second retransmission or second retry). Graphs 105-113 Correspond to re-transmissions (retries) of the same data packet (R4-R12). As shown by graph 102, when a communication environment between two devices experiences a 0.0025 bit-error-rate (0.25%) each time a new original data packet is transmitted, over a series of original data packet transmissions, the receiving device will experience a packet error rate that reaches nearly 68%. In other words, in an environment in which 0.25% of the bits received exhibit errors, this BER will swell such that approximately 68% of the original data packets will exhibit an error upon receipt (assuming a payload in each data packet of 48 bytes).

Each time a data packet is retransmitted, the effective packet error rate decreases even though the BER remains the same. By way of example, in a communications environment experiencing a 0.0025 bit-error-rate (0.25%), the packet error rate at the 5th retry is approximately 15% and at the 6th retry is approximately 10%. Returning to FIG. 1, this same correlation between BER and PER at the 5th and 6th retries (R5 and R6) can be seen at data points 126 and 127. Data points 126 and 127 correspond to the PER at the 5th and 6th retransmissions of a data packet in a communications environment experiencing a BER of 0.25%.

Previously, in the conventional approach, when a device received a data packet exhibiting errors, the device would discard/delete the “bad” data packet and send a response to the transmitting device indicating that the transmitting device should retry/retransmit the data packet. The conventional receiving device would then repeat the foregoing process until receiving an error-free data packet or reaching a time/count limit. The conventional device did not retain, nor pass on, the bad data packet for further processing. The conventional device would only pass on error-free data packets.

More recently, updates to the Bluetooth protocol have been proposed that utilize forward error correction (FEC). Forward error correction involves adding redundant bits to data packets, at the transmitting device, to help the decoder, at the receiving device, detect and correct some transmission errors without the need for retransmission. For example, in accordance with Bluetooth 5.0, three physical layers are provided to support communication, namely LE 1M, LE 2M and LE Coded (S=2 and S=8). The following Table 1 illustrates characteristics of the physical layers supported by Bluetooth 5.0. As noted in table 1, the LE Coded PHY offers forward error correction, but as a substantially lower data rate. Hence, a trade off exists between a higher data rate with no FEC (e.g., LE 1M at 1 Mbit/s or LE 2M at 2 Mbit/s) and a lower data rate with FEC (LE Coded S=2 has a data rate of 500 Kbit/s and LE Coded S=8 has a data rate of 125 Kbit/s).

TABLE 1

| | | LE | LE | |
|---|---|---|---|---|
| | | Coded | Coded | |
| | LE 1M | S = 2 | S = 8 | LE 2M |
| Symbol Rate | 1 | 1 | 1 | 2 |
| | Ms/s | Ms/s | Ms/s | Ms/s |
| Data Rate | 1 | 500 | 125 | 2 |
| | Mbit/s | Kbit/s | Kbit/s | Mbit/s |
| Error | CRC | CRC | CRC | CRC |
| Detection | | | | |
| Error | NONE | FEC | FEC | NONE |
| Correction | | | | |
| Range | 1 | 2 | 4 | 0.8 |
| Multiplier | | | | |
| (approx.) | | | | |
| Bluetooth 5 | Mandatory | Optional | Optional | Optional |
| Requirement | | | | |

### B. Improved Functioning of Computer and/or Communication Technology

In accordance with embodiments herein, a new and unique manner has been developed to improve the packet error rate for a given bit error rate through retransmission. Embodiments herein achieve substantially better packet error rates, even in the presence of poor bit error rates, and with substantially fewer retries for any given data packet. In accordance with new and unique aspects herein, improvements in the packet error rate are achieved in part by retaining and combining received data packets from the original and corresponding retries. For any given original data packet, the methods, devices and systems herein retain and combine at least a portion, if not all, of the bad versions of the data packet through the use of a conceptual, functional and/or physical candidate packet. Maintaining and updating the candidate packet in the various manners described herein afford a substantial improvement over existing computer and communications technology by effectively producing a corrected version of an original data packet separate and apart from forward error correction (if available). Among other improvements, embodiments herein reduce a number of retry data packets that are transmitted which among other things conserves power and extends battery life.

In accordance with embodiments herein, the original and retransmitted data packets are combined to form an error-free combination data packet. The error-free combination data packet is formed without regard for whether the data packet includes an FEC portion. For example, an error free combination data packet may be formed from an original and one or more retry data packets, none of which included forward error correction. Additionally or alternatively, even if the original and retry data packets included FEC content, the combination processes herein operation independent of and without regard for FEC content in the data packet. Thus, the processes herein may further improve the outcome of a protocol that utilizes FEC.

When a retry data packet is transmitted, statistically when the overall bit error rate is low, there is a very low probability that the retry data packet will have error(s) in the same bit location(s) as the error(s) in the original data packet. Similarly, when considering three data packets (e.g., original data packet, first retry and second retry data packet), statistically there is a lower probability that all three data packets will experience error(s) in the same bit location(s). Therefore, when any give bit location is compared between the three data packets (e.g., bit X in original data packet versus bit X in first retry data packet versus bit X in second retry data packet) there is a low probability that the bit value is wrong in all three data packets.

The present application recognizes that, even though, the location of an error in any given data packet is unknown, there is a high probability that the value is correct for a specific bit in most, if not all, of multiple retransmissions of a common data packet. For example, when the same data packet is transmitted three times, there is a very high probability that the value of any given bit X in at least two of the three data packets, is correct. In accordance with new and unique aspects herein, it has been recognized that, with retries, the equivalent failure bit error rate (FBER) at any given bit location in a data packet can be estimated based on select functional (e.g. logical or mathematical) combinations of the same bit X from the original and retry data packets. Non-limiting examples of the manner of mathematical combination include averaging, summing, determining the mode and the like. Nonlimiting examples of logical combinations include AND, OR, NOT OR, NOT AND, exclusive OR, and NOT exclusive OR multiple operators and the like.

Further, as an optional additional operation, it has been recognized that the FBER at any given bit location can be even further quantified by applying criteria to the functional combination. For example, when the functional combination represents a mathematical combination utilizing averaging, one or more threshold criteria may be applied (e.g., threshold=0.5) to round/adjust the average to a binary value. As a further example, when the average equals or is above 0.5, the process rounds/adjusts up the value of the averaged mathematical combination to 1. When the average is below 0.5, the process rounds/adjusts down the value of the averaged mathematical combination to 0. Alternatively, when the average is above 0.5, the process rounds/adjusts up the value of the averaged mathematical combination to 1. When the average equals or is below 0.5, the process rounds/adjusts down the value of the averaged mathematical combination to 0. Optionally, the average mathematical combination may be utilized without applying threshold criteria.

When the communications environment introduces a bit error rate of 0.0025, the corresponding failure bit error rate (FBER) with 2 retries is approximately 0.000019. Hence, the failure bit error rate is further reduced as compared to the conventional approach discussed above in connection FIG. 1. Thus, the present combination and criteria process (e.g., dynamic digital averaging) improves the communication quality without any sacrifice of the throughput as the retries will need to be carried out anyway. The present application improves upon base functioning of a computer and/or communication technology by evaluating i) good bits in the multiple retried error data packets against ii) the bad bits in the retried error data packets and by not discarding/deleting the data packets with bad bits without further analysis. The present application improves upon base functioning of a computer and/or communication technology by recognizing that, even in the error conditions mentioned, the number of the good bits outweighs the bad bits by a significant number.

FIG. 2 illustrates a collection of graphs representing simulations for a relation between the bit error rate and the transmitted packet error rate for a transmitted data packet (T1), re-transmitted (retried) packets (R3, R5, R6) and the packet error rate when applying the present processes to form an error corrected resultant data packet (DA3, DA5). The horizontal axis denotes a bit error rate that may be experienced by a communications environment. The vertical axis denotes a rate at which data packets conveyed through the communications environment exhibit errors upon receipt at a receiving device. The simulations of FIG. 2, as in the simulations of FIG. 1, assume transmission of data packets having 48 bytes of payload.

Graphs 202-216 individually correspond to the number of times that a common data packet is retransmitted. Graphs 202, 204, 206 and 207 correspond to graphs 102, 104, 106 and 107, respectively, in FIG. 1. Graph 202 corresponds to a new or original data packet (T1) transmitted for the first time. Graph 204 corresponds to a data packet (R3) that was previously transmitted, but upon arrival was found to exhibit an error and then was retransmitted twice before receiving an error-free retry (herein referred to as the second retransmission or second retry). Graph 204 corresponds to a data packet (R5) that was previously transmitted four times, upon prior arrivals was found to exhibit an error and then was retransmitted a fifth time before receiving an error-free retry. Graph 205 corresponds to a data packet (R6) that was previously transmitted five times, upon prior arrivals was found to exhibit an error and then was retransmitted a sixth time before receiving an error-free retry.

Graphs 215-216 correspond to simulations associated with the new processes implemented herein, to combine “bad” data packets until the resultant data packet is deemed to represent a corrected version of the original bad data packet. The packet error rates resulting from the new processes exhibit a substantial improvement over the packet error rates derived from the conventional approach. For example, as noted at data point is 226, when a communications environment experiences a 0.0025 bit-error-rate (0.25%), the new processes achieve a packet error rate of less than one percent (1%), with only 2 retries of the original data packet (3 transmissions in total), whereas, the conventional approach would exhibit a packet error rate of over 30% after two retries (R3) (see data point 228) and would exhibit a PER of around % 15 after four retries (R5) (see data point 229).

The improvements of the present processes over conventional approaches are beneficial in harsher communications environments. For example, as noted at data point is 236, when a communications environment experiences a 0.0035 bit-error-rate (0.35%), the new processes achieve a packet error rate of less than three percent (3%), with only 2 retries of the original data packet (3 transmissions in total), whereas, the conventional approach would exhibit a packet error rate of over 50% after two retries (R3) (see data point 238) and would exhibit a PER of % 30 after four retries (R5) (see data point 239). In view of the foregoing, it is clear the new processes afford substantial improvements upon base functioning of a computer and/or communication technology that merely applies retries of bad data packets.

Optionally, the mathematical combination of bad data packets may apply a process other than averaging, such as utilizing summing or determining a mode. When utilizing summing or determining the mode, the process may not apply threshold criteria. Alternatively, one or more threshold criteria may be applied to a sum-based or mode-based mathematical combination. For example, when utilizing a sum-based mathematical combination, the criteria may determine whether the sum is nearer a maximum potential sum (e.g., for 3 transmissions, nearer to 3 than to 0) or nearer a minimum potential sum (e.g., for 3 transmissions, nearer to 0 than to 3).

The following is an example of the primary flow steps with the error rates obtained with simulation. The simulation assumes a payload of 48 bytes. The initial packet error rate in an environment of bit error rate of 0.0025 is at 68.%. The initial packet error rate in an environment of bit error rate of 0.0250 is at ˜99.9 . . . %. If the initial packet has an error, with the 1st retry, the same packet is now sent and received twice. If the retry packet has no error, the loop complete and continue, otherwise, the retry packet error rate with the same bit error rate of 0.0025 is at 46.3%, and the retry packet error rate with the same bit error rate of 0.0250 is at ˜99.9%. The functional combination CPR (2) packet error rate with the bit error rate of 0.0025 is at 41.6%, and the functional combination CPR (2) packet error rate with the bit error rate of 0.025 is ˜99.9 . . . %.

If the 1st retry packet has an error, with the 2nd retry, the same packet is now sent and received 3 times. If the retry packet has no error, the loop complete and continue, otherwise, the retry packet error rate with the bit error rate of 0.0025 is now at 31.5%, and the retry packet error rate with the bit error rate of 0.0250 is at ˜99.9%. The functional combination CPR (3) packet error rate with the bit error rate of 0.0025 is <1.0%, and the functional combination CPR (3) packet error rate with the bit error rate of 0.025 is ˜57%.

If the 2nd retry packet has an error, with the 3rd retry, the same packet is now sent and received 4 times. If the retry packet has no error, the loop complete and continue, otherwise, the retry packet error rate with the bit error rate of 0.0025 is now at 21.5%, and the retry packet error rate with the bit error rate of 0.0250 is at ˜99.9 . . . %. The (4) packet error rate with the bit error rate of 0.0025 is <0.9%, and the functional combination CPR (4) packet error rate with the bit error rate of 0.025 is ˜42.5%.

If the 3rd retry packet has an error, with the 4th retry, the same packet is now sent and received 5 times. If the retry packet has no error, the loop complete and continue, otherwise, the retry packet error rate with the bit error rate of 0.0025 is now at 14.6%, and the retry packet error rate with the bit error rate of 0.0250 is at ˜99.9 . . . %. The functional combination CPR (5) packet error rate with the bit error rate of 0.0025 is <0.1%, and the functional combination CPR (5) packet error rate with the bit error rate of 0.025 is ˜<6%.

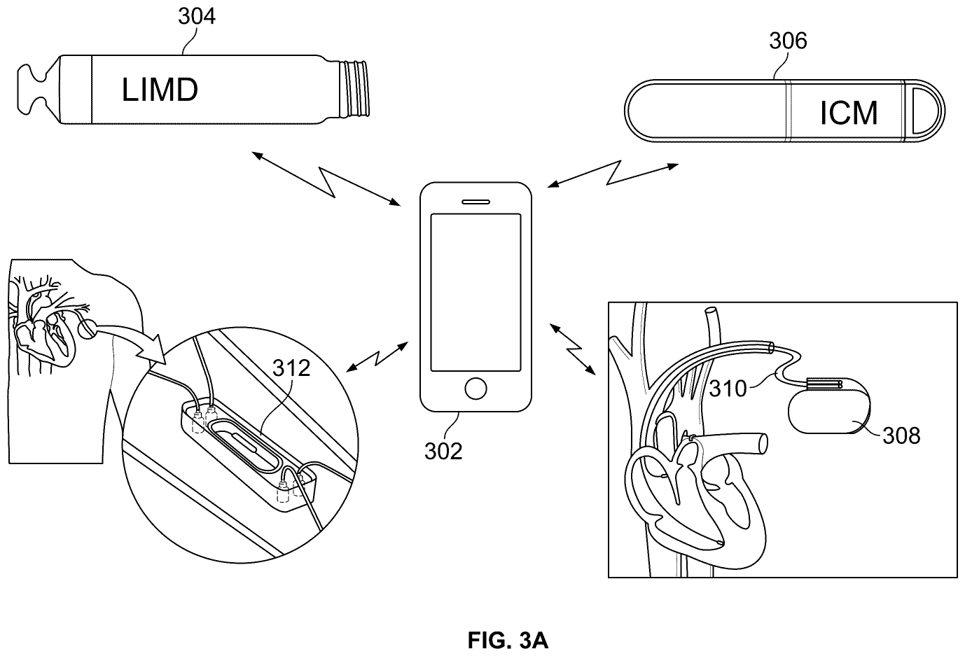

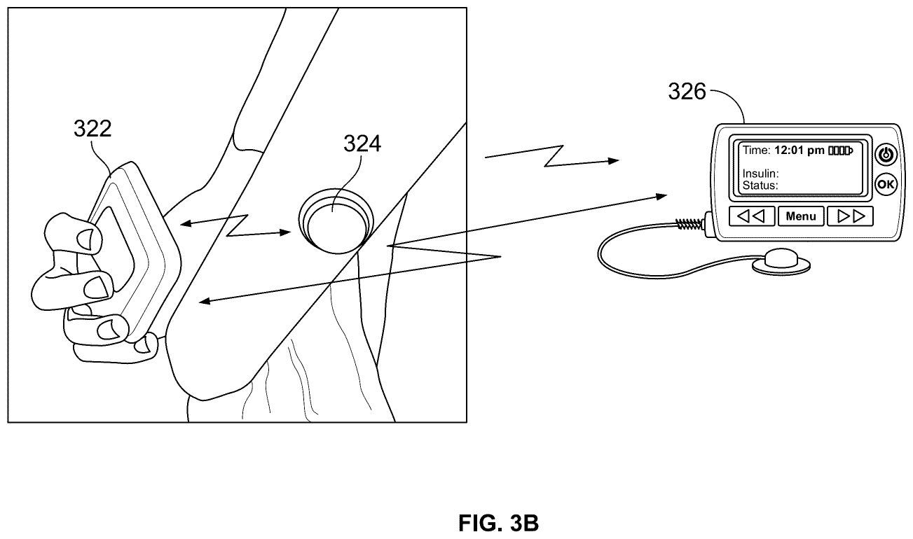

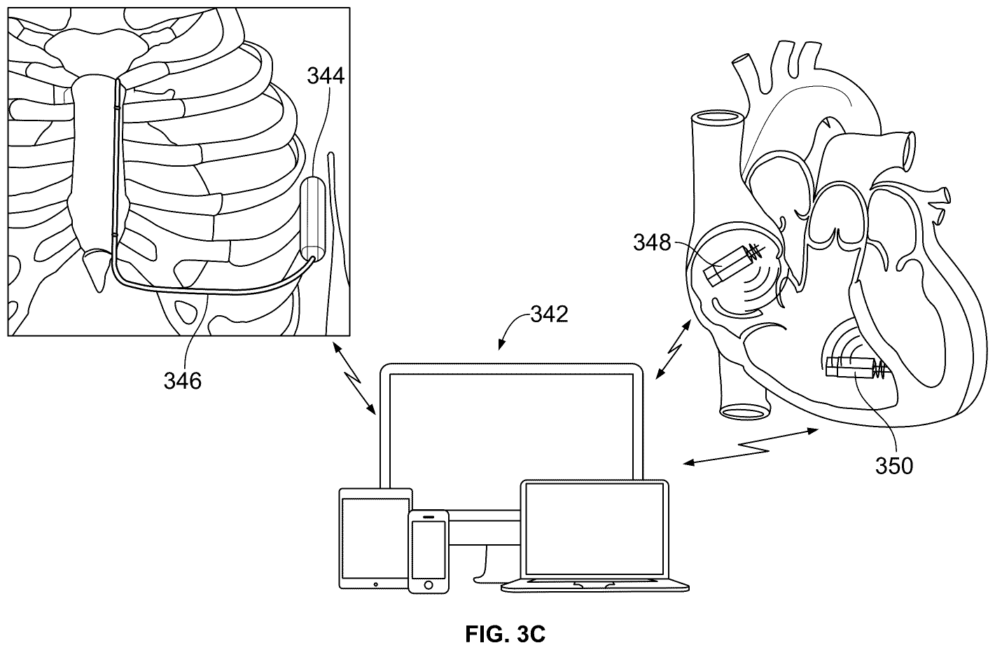

FIGS. 3A-3C illustrate examples of environments in which embodiments may be implemented. FIG. 3A illustrates an external device 302, such as a smart phone or other hand electronic device, configured to wirelessly communicate with one or more medical devices, such as one or more of the implantable medical devices illustrated. The external devices, wearable monitor, drug delivery pump and IMDs of FIGS. 3A-3C include, among other things, communications circuitry configured to communicate with one another utilizing one or more predefined communications protocols, examples of which are discussed herein. In the example of FIG. 3A, one or more of the implantable medical devices may be implanted in the same patient. For example, the patient may receive a leadless pacemaker 304, an implantable cardiac monitor 306, an implantable medical device 308 with one or more transvenous leads 310, and/or pulmonary arterial pressure monitor 312. In the event that more than one of the implantable medical devices 304-312 are utilized, the commonly implanted IMDs may further be configured to communicate with one another utilizing the same or a different communications protocol as used to communicate with the external device 302.

FIG. 3B illustrates a system to monitor an analyte such as glucose, where the system includes an external device 322, an analyte sensor 324 and, optionally, a drug delivery pump 326. FIG. 3C illustrates a system that includes implantable medical devices, one or more of which may be implanted alone or in combination in the same patient. Various external devices 342 may be configured to communicate with a subcutaneous IMD 344 coupled to one or more subcutaneous electrodes 346. Additionally or alternatively, the external device 342 may be configured to communicate with one or more leadless pacemakers 348, 350. Optionally, a single one of the leadless pacemakers 348, 350 may be implemented. Optionally, when more than one leadless pacemaker 348, 350 is implanted, the leadless pacemakers 348, 350 may be configured to communicate with one another.

Additionally or alternatively, any combination of the external, wearable and implantable devices illustrated in FIGS. 3A-3C may be utilized in combination with a common patient for a common patient population.

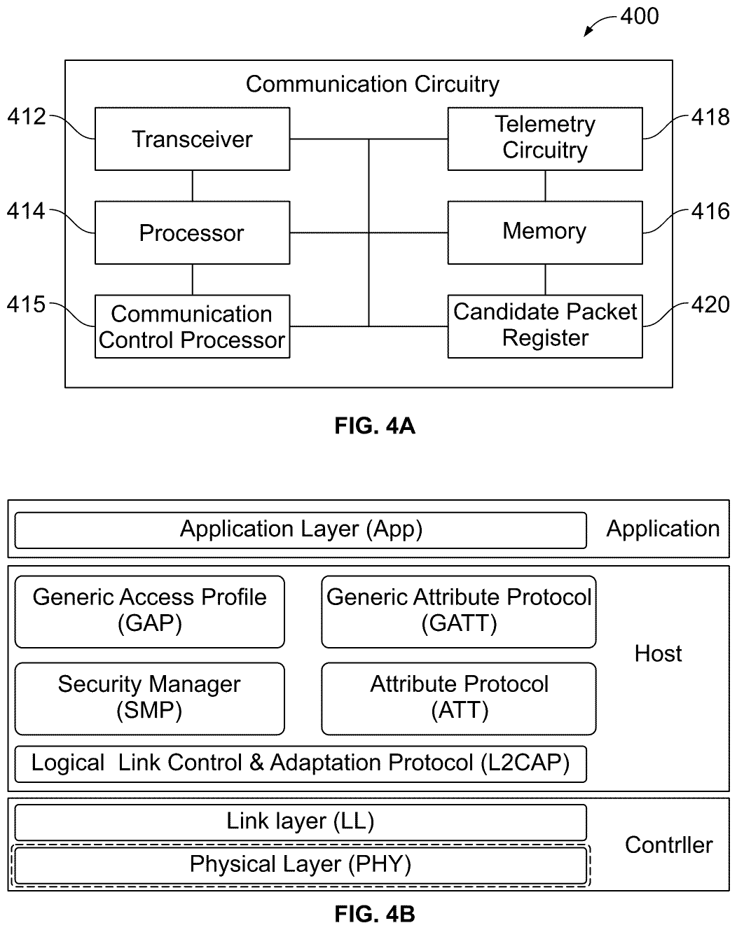

FIG. 4A illustrates a block diagram of communication circuitry 400 utilized in accordance with an embodiment herein. It is recognized that the blocks and interconnections shown in FIG. 4A are illustrative, not literal, and that the blocks and interconnections may be changed, merged, and otherwise modified. The components described herein can include or represent hardware and software instructions (e.g., software stored on a tangible and non-transitory computer readable storage medium, such as a computer hard drive, ROM, RAM, or the like) that perform the operations described herein. The hardware may include electronic circuits that include and/or are connected to one or more logic-based devices, such as microprocessors, processors, controllers, or the like. Additionally or alternatively, the components may be hard-wired logic circuits.

The communication circuitry 400 is within an IMD, a wearable monitor, a drug delivery pump, and/or one or more external devices. The communication circuitry 400 is configured to establish and maintain communication links between any combination of the implantable and/or wearable devices and external devices. In one example, the communication circuitry 400 may be configured to handle and/or manage bi-directional communication links. In one example, the communication circuitry 400 is an RF circuit. In another example, the communication circuitry 400 includes a transponder that transmits signals and a receiver that receives signals. In yet another example, the communication circuitry 400 includes a transceiver 412 (TX/RX) that both transmits signals and receives signals. Specifically, a transceiver includes both a transponder and a receiver. As explained herein, the communication circuitry 400 transmits, among other things, advertisement notices, connection requests, connection responses, scan requests, scan responses, data packets and the like. The transceiver 412 is tuned to communicate over one or more advertising and communications frequency bands in accordance with a corresponding protocol. Optionally, the communication circuitry 400 may be electrically coupled to an antenna (not shown).

The communication circuitry 400 includes one or more processors 414 including a communication controller 415, a local memory 416, and telemetry circuitry 418, all of which may be implemented on a common circuit board, within a common subsystem or within a common integrated circuit. Specifically, the communication circuitry 400 is in communication with other circuits, components, and modules of the other devices. The communication controller 415 may support one or more wireless communication protocols, such as Bluetooth low energy, Bluetooth, Medical Implant Communication Service (MICS), ZigBee, Wi-Fi, ultrawideband, inductive telemetry, near field communications (NFC) and the like.

Memory 416 stores instructions implemented by the communication controller 415. Protocol firmware may be stored in memory 416, which is accessed by the communication controller 415. The protocol firmware provides the wireless protocol syntax for the communication controller 415 to assemble data packets, advertisement notices, connection request data packets, connection responses, establish communication links and/or partition data received.

Communication circuitry 400 includes a candidate packet register (CPR) 420 that is utilized in connection with embodiments herein to afford improvements in computer and communications technology. For example, the candidate packet register may represent a portion of memory 416, a register within a BLE protocol stack (FIG. 4B), a separate portion of an integrated circuit, a buffer and the like. As explained herein, incoming data packets that exhibit an error are incorporated into the candidate packet register. The incoming data packets may be iteratively incorporated into the candidate packet register utilizing various functional combinations, such as mathematical combinations and/or logical combinations. The content of the candidate packet register may be further processed, such as by applying rounding or other operations. The content of the candidate packet register may be compared, on a bit-by-bit basis, to one or more threshold criteria that are then utilized to update the candidate packet register on a bit-by-bit basis. The content of the candidate packet register is then analyzed to determine whether it still exhibits one or more errors. Once the content of the candidate packet register passes an error detection process (e.g. is deemed to be error-free), the content of the register is declared to represent a resultant packet that is output from the communication circuitry as a corrected version of the original data packet in the form transmitted from the source device. By way of example, incoming data packets that exhibit errors (associated with a common original data packet) may be combined through a process referred to as dynamic digital averaging, as described further herein. Dynamic digital averaging represents one example of the manner in which data packets may be functionally combined within the candidate packet register.

The telemetry circuitry 418 in one example monitors various characteristics of the communications link. Alternatively, the telemetry software is stored in the memory that provides instructions that are followed by the communication control processor or one or more of the processors 414 related to the communication circuitry 400. The telemetry circuitry 418 in one example determines when the link drops, and/or how often the link drops causing interruptions in communications being passed through the communications link. In another example the telemetry circuitry 418 determines the number of return errors received, number of bad data packets received, and the like. Optionally, telemetry circuitry 418 tracks the signal to noise ratio (RSSI) of the communication link. When the communication link is down or not working the telemetry circuitry 418 verifies the power setting of the telemetry link and increases the power setting when a break in the link occurs, or when a link is unable to be established. In this manner, the telemetry circuitry 418 facilitates re-establishment of the link to assist in completing communication sessions. Thus, the telemetry circuitry 418 not only makes determinations regarding when communication breaks occur including timing to reestablish the link and the like, but additionally, the quality of the telemetry link is determined through various methods including RSSI. Telemetry circuitry 418 also actively corrects any breaks or poor-quality communications by increasing power to establish and maintain the link.

FIG. 4B illustrates an example protocol stack structure of a Bluetooth protocol. The top layer corresponds to various applications that interact with and utilize Bluetooth wireless communication. The host maintains various information, such as the generic access profile (GAP), generic attribute protocol (GATT), secure manager (SMP), attribute protocol (ATT), and logical Link control and adaptation protocol (L2CAP). The controller implements, among other things, the link layer (LL) and the physical layer (PHY).

Returning to FIG. 4A, the communications circuitry 400 is configured to implement the protocol stack structure of FIG. 4B, including one or more physical layers. For example, the communications circuitry 400 may utilize a single physical layer as in certain versions of the Bluetooth protocol or multiple physical layers as in new versions of the Bluetooth protocol. For example, when more than one physical layer is available, the communications circuitry 400 may be configured to selected one or a combination of the LE 1 M, LE 2 M, LE Coded physical layers (including the LE Coded 2S and LE Coded 8S). Optionally, during a communications session, the communications circuitry 400 may transmit utilizing a first PHY and receive utilizing a second PHY. For example, during a communications session in which the IMD is downloading a new firmware version, the IMD may receive data utilizing the LE 2 M PHY, while utilizing one of the LE Coded PHY to transmit to the ED. As another example, during a communication session in which the IMD is uploading stored EGM signals, the IMD may utilize the LE 2 M PHY to transmit the EGM signals to the ED, but utilize one of the LE Coded PHY to receive commands from the ED.

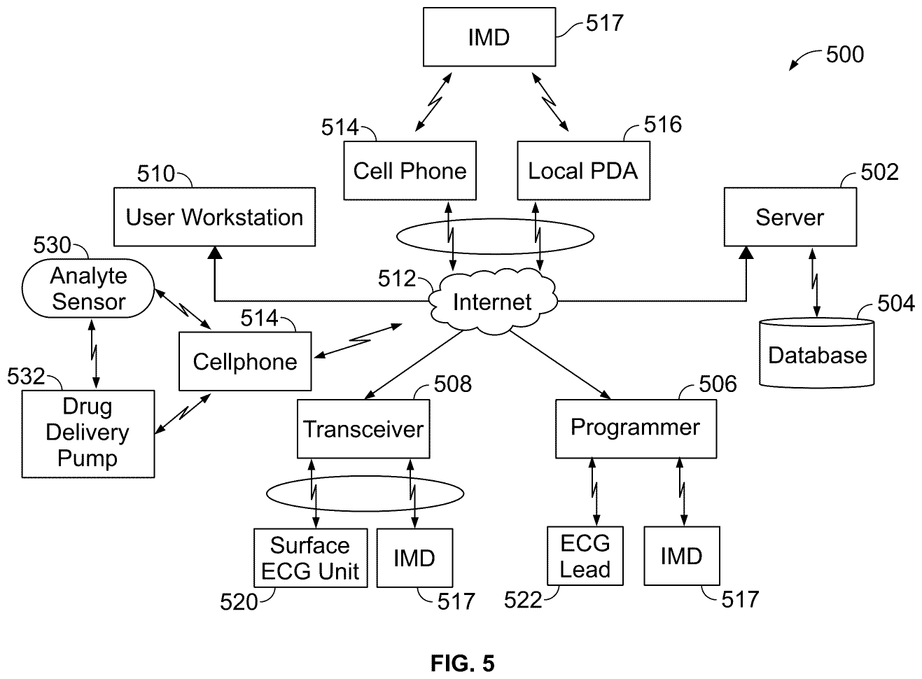

FIG. 5 illustrates a distributed processing system in accordance with one embodiment. In one example, the distributed processing system 500 includes and implements the communication circuitry 400 as provided in FIG. 4A in one or more of the devices shown in FIG. 5. All of the communications links shown in FIG. 5 are bidirectional. The distributed processing system 500 includes a server 502 connected to a database 504, a programmer 506, a local RF transceiver 508 and a user workstation 510 electrically connected to a communication system 512. Any of the processor-based components in FIG. 5 (e.g., workstation 510, cell phone 514, PDA 516, server 502, programmer 506, IMD 501) may communicate wirelessly with IMD 517. In one example, the local RF transceiver 508 is the transceiver of 412 of FIG. 4A. The various components illustrated in FIG. 5 are configured to communicate over a common wireless protocol, such as the Bluetooth low energy protocol. The components may implement various versions of the BLE protocol. For example, a subset of the IMDs 517 may implement a version 4.0 or lower version of the BLE protocol (e.g., having only the LE 1 M PHY), while another subset of the IMD 517 may implement a version 5.0 or higher version of the BLE protocol (e.g., having multiple PHYs). The cell phone 514 and programmer 506 may include transceivers that support multiple PHYs (e.g., LE 1 M, LE2 M, LE coded), while the local PDA 516 only supports the LE 1 M PHY.

The communication system 512 may be the internet, a voice over IP (VoIP) gateway, a local plain old telephone service (POTS) such as a public switched telephone network (PSTN), a cellular phone-based network, and the like. Alternatively, the communication system 512 may be a local area network (LAN), a campus area network (CAN), a metropolitan area network (MAN), or a wide area network (WAN). Communication system 512 serves to provide a network that facilitates the transfer/receipt of information such as cardiac signal waveforms, ventricular and atrial heart rates.

The server 502 is a computer system that provides services to other computing systems over a computer network. The server 502 interfaces with the communication system 512 to transfer information between the programmer 506, the local RF transceiver 508, the user workstation 510 as well as a cell phone 514, a personal data assistant (PDA) 516, and IMD 517 to the database 504 for storage/retrieval of records of information. On the other hand, server 502 may upload raw cardiac signals from an implanted lead 522, surface ECG unit 520 or the IMD 517 via the local RF transceiver 508 or the programmer 506.

Database 504 stores information such as cardiac signal waveforms, ventricular and atrial heart rates, IMD data, BGA data, thresholds, and the like, for a single or multiple patients. The information is downloaded into database 504 via server 502 or, alternatively, the information is uploaded to the server from database 504. The programmer 506 is similar to an external device or instrument and may reside in a patient's home, a hospital, or a physician's office. Programmer 506 interfaces with the lead 522 and the IMD 517. The programmer 506 may wirelessly communicate with the IMD 517 and utilize protocols, such as Bluetooth, GSM, infrared wireless LANs, HIPERLAN, 3G, satellite, inductive as well as circuit and packet data protocols, and the like. Alternatively, a hard-wired connection may be used to connect the programmer 506 to the IMD 517. The programmer 506 is able to acquire cardiac signals from the surface of a person (e.g., ECGs), intra-cardiac electrogram (e.g., IEGM) signals from the IMD 517, and/or cardiac signal waveforms, ventricular and atrial heart rates, and detection thresholds from the IMD 517. Programmer 506 interfaces with the communication system 512, either via the internet or via POTS, to upload the information acquired from the surface ECG unit 520, the lead 522 or the IMD 517 to the server 502.

The local RF transceiver 508 interfaces with the communication system 512 to transmit and receive telemetry data and information being transmitted to and from the RF transceiver 508. In one example the communication system monitors the communication link and records telemetry breaks, length of breaks, number of breaks in a given interval, time to reestablish a signal, signal to noise ratio, and the like. In this manner the communication system 512 provides with a user the quality and/or strength of a communication signal and amount or strength of local interference. In addition, the communication system 512 (e.g., server 502, cell phone 514, workstation 510, programmer 506) may, based on the monitored communication quality, increase or decrease the power of a signal being transmitted. In one example, communication system 512 monitors both advertising channels and connection channels, thus providing advertising data packets through and communicating through the advertising channels with external devices. Similarly, the communication system 512 is able to provide a communication pathway through a connection channel.

The user workstation 510 may interface with the communication system 512 to download IMD data and/or BGA data including, but not limited to, cardiac signal waveforms, ventricular and atrial heart rates, and detection thresholds via the server 502 from the database 504. Alternatively, the user workstation 510 may download raw data from the surface ECG units 520, lead 522 or IMD 517 via either the programmer 506 or the local RF transceiver 508 or cell phone 514 or PDA 516. Once the user workstation 510 has downloaded the cardiac signal waveforms, ventricular and atrial heart rates, or detection thresholds, the user workstation 510 may process the information in accordance with one or more of the operations described above. The user workstation 510 may download the information and notifications to the cell phone 514, the PDA 516, the local RF transceiver 508, the programmer 506, or to the server 502 to be stored on the database 504.

### C. Embodiments for Implementing Improvement in Computer and/or Communication Technology

Next the discussion turns to example embodiments for implementing the improved processes and technology discussed herein. It should be noted that, while embodiments herein are described in connection with various versions of the Bluetooth low energy (BLE) communications protocol, the present application is not limited to BLE protocols. More generally, embodiments of the present application may be implemented in connection with non-BLE communications protocols that provide for a transmitting device to re-transmit data packets when the original received data packet included errors as received at the receiving device.

For example, the Zigbee protocol may be utilized. Zigbee is a wireless technology that enables low-power, low-data-rate, and low-cost communication between devices. Zigbee is based on the IEEE 802.15.4 standard and operates in the 2.4 GHz band. Zigbee is mainly used for IoT applications, such as smart home, smart lighting, smart metering, and industrial automation. Zigbee supports mesh networking, which allows devices to relay messages to each other and extend the network coverage. The Zigbee 2007 is the original version of Zigbee, which supports data rates of up to 250 kbps and is mainly used for home automation, lighting control, and security applications. Some of the profiles that Zigbee 2007 supports are Home Automation Profile (HAP), Commercial Building Automation Profile (CBAP), and Smart Energy Profile (SEP). The Zigbee PRO is an enhanced version of Zigbee, which supports data rates of up to 250 kbps and offers improved security, reliability, and network management. Zigbee PRO is mainly used for industrial, commercial, and residential applications. Some of the profiles that Zigbee PRO supports are Zigbee Light Link (ZLL), Zigbee Green Power (ZGP), and Zigbee IP (ZIP). The Zigbee 3.0 is the latest version of Zigbee, which supports data rates of up to 250 kbps and offers backward compatibility with previous Zigbee versions and profiles. Zigbee 3.0 is designed to unify the Zigbee ecosystem and enable interoperability among Zigbee devices from different manufacturers and applications.

Additionally or alternatively, the Z-Wave protocol may be utilized. Z-Wave also operates in the industrial, scientific, and medical (ISM) radio bands. Z-Wave devices can use the 800-900 MHz frequency band, which has 16 channels and a data rate of up to 100 kbps. Z-Wave devices can transmit data over distances of up to 100 meters indoors and 800 meters outdoors, depending on the power output and environmental conditions. Z-Wave devices can communicate in different modes, such as point-to-point, broadcast, and mesh, and is compatible with the Internet Protocol (IP).

Additionally or alternatively, the Wi-Fi protocol may be utilized. Wi-Fi typically operates within a range of up to 100 meters indoors and up to 300 meters outdoors, depending on the power and version of the Wi-Fi device. However, Wi-Fi range can be affected by various factors, such as obstacles, interference, antenna design, and environmental conditions. Examples of Wi-Fi versions that may be utilized include: Wi-Fi 4 (IEEE 802.11n), Wi-Fi 5 (IEEE 802.11ac), Wi-Fi 6 (IEEE 802.11ax), or Wi-Fi 7 (the next-generation wireless standard). Wi-Fi 7, officially known as 802.11be, builds on the foundation laid forth by Wi-Fi 6E.

Additionally or alternatively, the Ultra-Wideband (UWB) protocol may be utilized. UWB is a wireless technology that uses very low energy pulses of radio waves to transmit data and measure the location and direction of objects with high accuracy. UWB can operate over a large portion of the radio spectrum, typically from 3.1 GHz to 10.6 GHz, and can co-exist with other wireless technologies, such as Wi-Fi, Bluetooth, and NFC, without causing interference. Additionally or alternatively, Near-Field Communication (NFC) may be utilized.

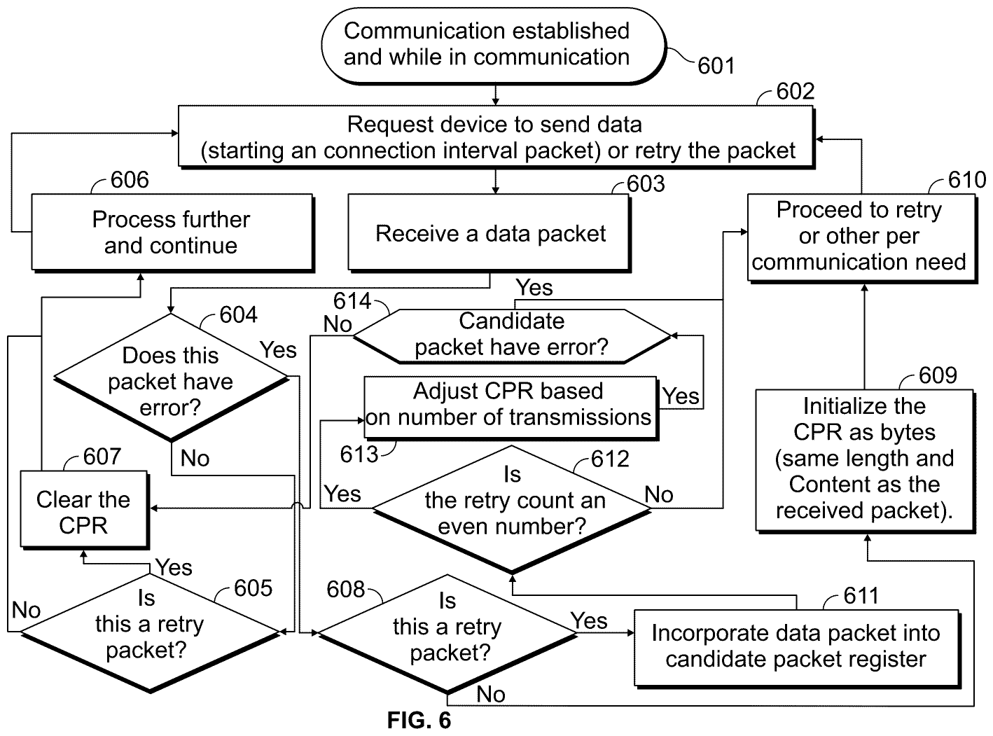

FIG. 6 illustrates a method for managing communication between at least first and second devices in accordance with embodiments herein. FIG. 6 is from the perspective of a first device operating as a “master device” that is communicating with a second device that is operating as a “slave” device. For example, the first/master device may represent an external device (e.g., cell phone, programmer, tablet device, bedside monitor, wearable drug delivery device), while the second/slave device may represent a medical device (e.g., an IMD, a wearable analyte sensor, a smart watch, a drug delivery pump). Alternatively, the second/slaver device may represent the external device (e.g., cell phone, programmer, tablet device, bedside monitor, wearable drug delivery device), while the first/master device may represent the medical device (e.g., an IMD, a wearable analyte sensor, a smart watch, a drug delivery pump). Additionally or alternatively, the first/master and second/slave devices may both represent medical devices (e.g., two IMDs, an analyte sensor and a drug delivery pump, an IMD and analyte sensor, an IMD and a drug delivery pump).

The operations of FIG. 6 may be implemented in whole or in part within communication circuitry such as by the communication circuitry 400. Optionally some or all of the operations of FIG. 6 may be implemented by one or more processors, such as one or more processors within the communication circuitry and/or one or more processors more generally within the corresponding device.

At 601, the first/master and second/slave devices establish a communications link in accordance with a predetermined protocol. For example, the first and second devices may establish a communications link utilizing one of the various versions of Bluetooth (e.g., Version 4, Version 4.2, Version 5, Version 5.4). Optionally, the communications link may be established in connection with the ZigBee protocol, Wi-Fi protocol, ultrawideband protocol, inductive telemetry, near field communications (NFC) protocol and the like.

At 602, the communication circuit of the first device sends to the communication circuit of the second device, a request or instruction for the second device to start sending data. For example, the first device may represent an external device, such as a smart phone or bedside monitor, that sends a request for real-time or stored data from an IMD, wearable analyte patch, drug delivery pump and the like. The data may represent stored or real time IMD data, BGA data and/or BRM data. The second/transmitting device begins sending data packets during one connection interval or a series of successive connection intervals, during which the subsequent operations at 603-614 are implemented. As one example, the operations at 603-614 may be implemented during a single connection interval before termination of the current single connection interval.

At 603, a transceiver of the first device receives a data packet. The data packet includes, among other things, a header block, a payload block and an error detection block. For example, the error detection block may represent a cyclic redundancy check (CRC) code generated, based on the content of the payload block and added to the data packet before transmission from a slave/second device (also referred to as a source or transmitting device). CRC error detection is one example. Optionally, embodiments herein may implement other types of error detection, such as parity bit, check sum, repeat data, cryptographic hash functions and the like. Additionally or alternatively, the error detection block may implement forward error correction (FEC), such as utilizing convolutional codes, block codes and the like. The data packet may represent an original data packet that includes payload content transmitted for the first time from the second/slave device. Alternatively, the data packet represents a retry data packet transmitted from the slave/second device with the same payload content as previously transmitted in a prior corresponding original data packet and/or prior corresponding retry data packet.

At 604, the first device determines whether the data packet exhibits an error. For example, the communication circuitry (e.g., integrated circuits and/or one or more processors) of the first device may unpack the data packet and identify the error detection block and the payload block within the data packet. The communication circuitry recalculates the error detection value associated with the payload block and compares the newly calculated error detection value to the content of the incoming error detection block. When the newly calculated error detection value matches the content of the error detection block, the communication circuitry designates the data packet to be true or correct. When the newly calculated error detection value does not match the content of the error detection block, the communication circuitry designates the data packet to be bad or to exhibit an error. When the data packet does not exhibit an error, the data packet is considered to be error-free and flow moves to 605.

At 605, the communication circuitry determines whether the data packet is a “retry” data packet or a “new” or “original” data packet. When the data packet is a new or original data packet, flow moves to 606. At 606, the new/original data packet is passed on to be stored or further processed within or outside of the communication circuitry. When the data packet is a retry data packet, flow moves to 607.

At 607, the communication circuitry clears the candidate packet register (CPR). For example, the CPR may represent a physical register such as a buffer, but with the cells or fields in the register not necessarily limited to a binary structure. For example, each cell in the CPR will correspond to a bit of the incoming data packet, but the cells of the CPR may have values greater then “1”. Additionally or alternatively, the CPR may merely represent a conceptional or functional portion of memory, of the BLE protocol stack, a separate portion of an integrated circuit, and the like. When implementing a BLE based protocol, the CPR may be implemented within or outside of BLE communication circuitry. As explained herein, each time an incoming data packet (original or retry) is determined to exhibit an error, the data packet is incorporated into the CPR. The data packet may be incorporated into the CPR through various function combinations (e.g. mathematical or logical combinations) and with or without the additional application of a threshold criteria. Maintaining and updating the candidate packet in the various manners described herein afford a substantial improvement over existing computer and communications technology by effectively producing a corrected version of an original data packet separate and apart from forward error correction (if available). At 607, the prior content of the CPR is cleared because the most recently received data packet was determined to not exhibit an error (be error free).

Thereafter, flow moves to 606 and the retry data packet is passed on to be stored or further processed within or outside of the communication circuitry. In the present embodiment, each data packet includes among other things, a header block, a payload block, and an error detection block or (optionally) an error correction block.

Returning to 604, when the communication circuitry determines that the data packet exhibits an error, flow moves to 608. At 608, the communication circuitry determines whether the data packet is a retry data packet or a new/original data packet. When the data packet is a retry data packet, flow moves to 609. At 609, the communication circuitry initializes the CPR by defining the length and content of the CPR to be the same as the length and content of the data packet determined to exhibit the error. For example, when the data packet includes a 2-byte error detection block (e.g., for CRC-16) and a 48-byte payload block, the CPR is initialized to include the same corresponding 2-byte error detection block and 48-byte payload block. Optionally, the CPR may also include all or a portion of any header from the data packet and/or any other protocol specific content in the data packet, preceding or following the payload block.

Thereafter flow moves to 610, the communication circuitry of the first device initiates a retry operation. For example, the first device may either not respond to the incoming data packet or respond with a not-acknowledge (NAC) response indicating that the most recent received data packet exhibited one or more errors. When the second device receives an NAC response or receives no response, the condition is interpreted to indicate that the previously transmitted data packet exhibited an error at reception, and the second device proceeds to retransmit the same payload content in a retry data packet.

Flow returns to 602 where the first device receives a retry data packet (responsive to the NAC response or lack of response at 610). Flow again moves through 603 to 604 where the retry data packet is tested again for errors. When the retry data packet exhibits an error, flow moves to 608. During this iteration (second iteration), the data packet is a retry data packet and thus flow moves to 611.

At 611, the communication circuitry incorporates the retry data packet into the CPR utilizing one or more function combinations (e.g. mathematical or logical) as described herein. As one example, the retry data packet may simply be added to the CPR on a cell-by-cell basis. For example, if the CPR content was 00101101 and the retry data packet content was 00101110, then the new CPR content would be 00202211. In this example, the content of the CPR does not necessarily maintain a “binary” structure, but instead the values for each cell or field in the CPR may exceed “0” and “1”, at least until subsequently averaged and reset to a binary value, as described herein. Optionally, the retry data packet may be incorporated into the CPR based on other mathematical and/or logical combinations. Non-limiting examples of the manner of mathematical combination include averaging, summing, determining the mode and the like. Nonlimiting examples of logical combinations include AND, OR, Not OR, Not AND, exclusive OR, Not exclusive OR multiple operators and the like.

At 612, the communication circuitry determines the number of times that a particular data packet has been “retried” and whether the retry count is even. For example, when the current iteration through FIG. 6 corresponds to a first retry data packet that followed a prior iteration in which the original data packet exhibited an error, the current retry count would be one, namely odd. Alternatively, when the current iteration through FIG. 6 corresponds to a second retry, following a first retry data packet and an original data packet that both exhibited an error, the current retry count is 2 and even. When the retry count is odd, flow skips to 610 where another retry operation is initiated (e.g. by responding with an NAC response for no response). When the retry count is even, flow moves from 612 to 613.

At 613, the communication circuitry modifies the CPR cell by cell to account for the number of iterations during which the CPR has been updated in connection with the same original payload. For example, if the current iteration is the third time that a single payload has been transmitted (e.g. in the original data packet followed by 2 retry data packets), then at 613, the candidate data packet is updated based on 3 iterations. As a further example, when the type of functional combination applied to the CPR is averaging, then at 613, the modification includes rounding the content of the CPR cell by cell based on the number of iterations in which the current data packet has been transmitted (including the original data packet and all retry data packets). For example, if the current iteration is the third time that a single payload has been transmitted (e.g. the original followed by 2 retries), then at 613, the CPR is divided by 3 cell by cell.

As a further example, when the functional combination represents a mathematical combination utilizing averaging, one or more logical criteria may be applied (e.g., threshold=0.5) to round/adjust the average to a binary value. As a further example, when the average equals or is above 0.5, the process rounds/adjusts up the value of the averaged mathematical combination to 1. When the average is below 0.5, the process rounds/adjusts down the value of the averaged mathematical combination to 0. Alternatively, when the average is above 0.5, the process rounds/adjusts up the value of the averaged mathematical combination to 1. When the average equals or is below 0.5, the process rounds/adjusts down the value of the averaged mathematical combination to 0. Optionally, the average mathematical combination may be utilized without applying threshold criteria.

Next flow moves to 614. At 614, the communication circuitry reapplies the error detection process to the present payload block, within the CPR, to obtain an error detection value and determines whether the content of the CPR includes one or more errors (also referred to as a CPR error). The communication circuitry compares the current CPR error detection value to the content of the error detection portion within the CPR. For example, the communication circuitry may apply a CRC code detection process to the payload portion the CPR to obtain a current CRC value. The current CRC value is compared to the CRC content of the error detection portion within the CPR. The analysis at 614, with respect to the CPR substantially corresponds to the original error detection process at 604, applied with respect to each incoming data packet. When the currently calculated error detection value matches the content of the error detection block within the CPR, the communication circuitry determines that the CPR corresponds to a valid or corrected version of the original payload transmitted from the second/slave device. When the payload block within the CPR is determined to be correct, the content is output as a “resultant” packet representing a corrected version of the payload block of the original data packet. When the payload block within the CPR is determined still to exhibit an error, flow moves to 610 where the communication circuitry implements another retry.

Additionally or alternatively, the decision at 612 may be omitted (the process does not need to check for an even number of retries), such as when the computation power consumption is sufficient/acceptable. When 612 is removed, flow would pass from 611 to 612 each iteration.

Additionally or alternatively, all or a portion of the operations of FIG. 6 may be implemented by one or more processors outside of the communications circuitry. For example, the operations at 601-603 and 610 may be implemented by the communications circuitry, while the remainder of the operations 604-609 and 611-614 may be implemented by circuitry and/or one or more processors that are connected to the communication circuitry.

Additionally or alternatively, the retry data packet may be incorporated into the CPR and the CPR tested for errors utilizing sequence analysis. Sequence analysis may further reduce the probability of error, because if the noise is purely random, the received retry data packet should have a bias towards the original data packet content.

Alternatively, as noted above, the operations of FIG. 6 may be implemented where the second device represents the external device (e.g., cell phone, programmer, tablet device, bedside monitor, wearable drug delivery device), while the first device represents the medical device (e.g., an IMD, a wearable analyte sensor, a smart watch, a drug delivery pump).

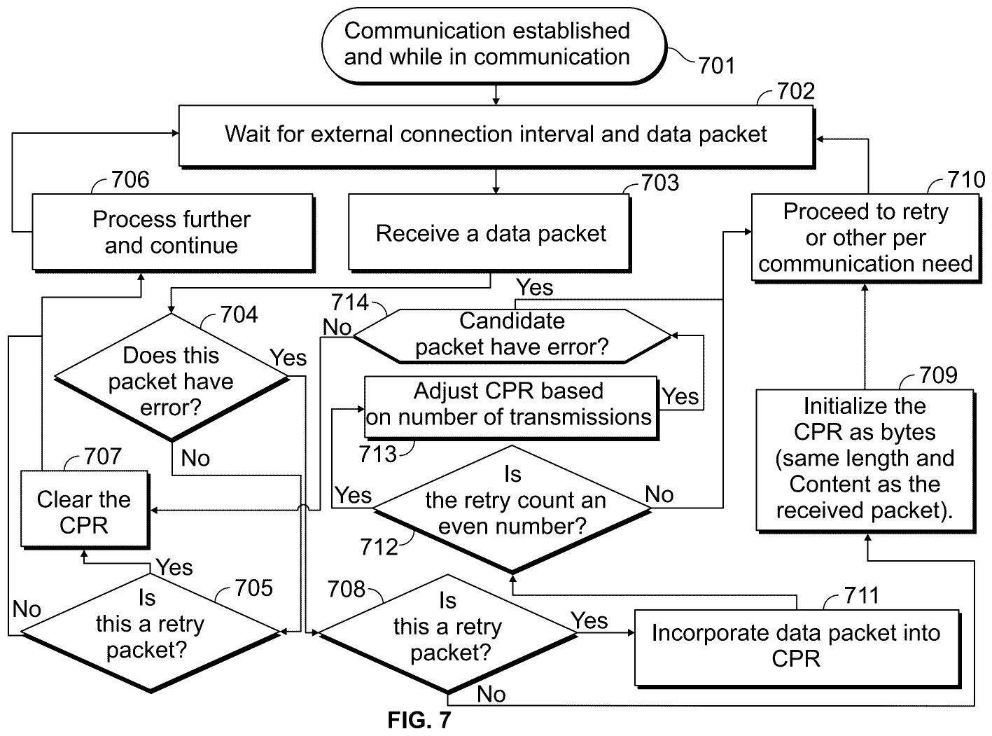

FIG. 7 illustrates a method for managing communication between at least first and second devices in accordance with embodiments herein. FIG. 7 is from the perspective of a second device operating as a “slave device” that is communicating with a first device that is operating as a “master” device. The operations of FIG. 7 substantially parallel the operations of FIG. 6, but from the perspective of a slave device. The first/master device may still represent an external device (e.g., cell phone, programmer, tablet device, bedside monitor, wearable drug delivery device), while the second/slave device may still represent a medical device (e.g., an IMD, a wearable analyte sensor, a smart watch, a drug delivery pump), or vice versa. Additionally or alternatively, the first/master and second/slave devices may both still represent medical devices (e.g., two IMDs, an analyte sensor and a drug delivery pump, an IMD and analyte sensor, an IMD and a drug delivery pump).

The operations of FIG. 7 may be implemented in whole or in part within communication circuitry such as by the communication circuitry 400. Optionally some or all of the operations of FIG. 7 may be implemented by one or more processors, such as one or more processors within the communication circuitry and/or one or more processors more generally within the corresponding device.

At 701, the first and second devices establish a communications link in accordance with a predetermined protocol. At 702, the communication circuit of the second device waits for an external connection interval and for an incoming data packet. For example, the first device may represent an external device, such as a smart phone or bedside monitor, that sends reprogramming or configuration data to an IMD, wearable analyte patch, drug delivery pump and the like. The second/transmitting device begins receiving data packets during one connection interval or a series of successive connection intervals, during which the subsequent operations at 703-714 are implemented. As one example, the operations at 703-714 may be implemented during a single connection interval before termination of the current single connection interval.

At 703, a transceiver of the second device receives a data packet. The data packet includes, among other things, a payload block and an error detection block. Additionally or alternatively, the error detection block may implement forward error correction (FEC), such as utilizing convolutional codes, block codes and the like. The data packet may represent an original data will packet that includes payload content transmitted for the first time from the first/master device. Alternatively, the data packet represents a retry data packet transmitted with the same payload content as previously transmitted in a prior corresponding original data packet and/or prior corresponding retry data packet.

At 704, the second device determines whether the data packet exhibits an error in the same manner as discussed above in connection with FIG. 6 and elsewhere. When the data packet does not exhibit an error, the data packet is considered to be error-free and flow moves to 705. At 705, the communication circuitry determines whether the data packet is a “retry” data packet or a “new” or “original” data packet. When the data packet is a new or original data packet, flow moves to 706. At 706, the new/original data packet is passed on to be stored or further processed within or outside of the communication circuitry. When the data packet is a retry data packet, flow moves to 707.

At 707, the communication circuitry clears the CPR. For example, the CPR may represent a physical register such as a buffer. Additionally or alternatively, the CPR may merely represent a conceptional or functional portion of memory, of the BLE protocol stack, a separate portion of an integrated circuit, and the like. When implementing a BLE based protocol, the CPR may be implemented within or outside of BLE communication circuitry. At 707, the prior content of the CPR is cleared because the most recently received data packet was determined to not exhibit an error (be error free). Thereafter, flow moves to 706 and the retry data packet is passed on to be stored or further processed within or outside of the communication circuitry.

Returning to 704, when the communication circuitry determines that the data packet exhibits an error, flow moves to 708. At 708, the communication circuitry determines whether the data packet is a retry data packet or a new/original data packet. When the data packet is a retry data packet, flow moves to 709. At 709, the communication circuitry initializes the CPR by defining the length and content of the CPR to be the same as the length and content of the data packet determined to exhibit the error. Thereafter flow moves to 710, the communication circuitry of the second device initiates a retry operation. For example, the second device may either not respond to the incoming data packet or respond with a not-acknowledge (NAC) response indicating that the most recent received data packet exhibited one or more errors. When the first device receives an NAC response or receives no response, the condition is interpreted to indicate that the previously transmitted data packet exhibited an error, and the first device proceeds to retransmit the same payload content (and error detection content) in a retry data packet.

Flow returns to 702 where the second device receives a retry data packet (responsive to the NAC response or lack of response at 710). Flow again moves through 703 to 704 where the retry data packet is tested again for errors. When the retry data packet exhibits an error, flow moves to 708. During this iteration (second iteration), the data packet is a retry data packet and thus flow moves to 711. At 711, the communication circuitry incorporates the retry data packet into the CPR utilizing one or more function combinations (e.g. mathematical or logical) as described herein. At 712, the communication circuitry determines the number of times that a particular data packet has been “retried” and whether the retry count is even. When the retry count is odd, flow skips to 710 where another retry operation is initiated (e.g. by responding with an NAC response for no response). When the retry count is even, flow moves from 712 to 713. At 713, the communication circuitry modifies the CPR cell by cell to account for the number of iterations during which the CPR has been updated in connection with the same original payload.

At 714, the communication circuitry reapplies the error detection process to the present payload block, within the CPR, to obtain an error detection value and determines whether a content of the CPR includes one or more errors (a CPR error). The communication circuitry compares the current error detection value to the content of the error detection block within the CPR. The analysis at 714, with respect to the CPR substantially corresponds to the original error detection process at 704, applied with respect to each incoming data packet. When the currently calculated error detection value matches the content of the error detection block within the CPR, the communication circuitry determines that the CPR corresponds to a valid or corrected version of the original payload transmitted from the second/slave device. When the payload block within the CPR is determined to be correct, the content is output as a “resultant” packet representing a corrected version of the payload block of the original data packet. When the payload block within the CPR is determined still to exhibit an error, flow moves to 710 where the communication circuitry implements another retry.

Additionally or alternatively, the decision at 712 may be omitted (the process does not need to check for an even number of retries), such as when the computation power consumption is sufficient/acceptable. When 712 is removed, flow would pass from 711 to 712 each iteration. Additionally or alternatively, all or a portion of the operations of FIG. 7 may be implemented by one or more processors outside of the communications circuitry. For example, the operations at 701-703 and 710 may be implemented by the communications circuitry, while the remainder of the operations 704-709 and 711-714 may be implemented by circuitry and/or one or more processors that are connected to the communication circuitry.

The embodiments of FIGS. 6 and 7 are described in the context of communications occurring during a communications session after a one-to-one communications link has been established. Optionally, the operations of FIGS. 6 and 7 may be implemented without ever establishing a one-to-one communications session. For example, embodiments may be implemented in which the device performing the operations of FIGS. 6 and 7 is operating as an “observer” device that is listening over advertisement channels for broadcasts from a transmitting device that is operating as a broadcasting device. For example, in accordance with the Bluetooth version 5.4, a new feature referred to as periodic advertising with response (PAwR) is provided. PAwR allows devices that receive periodic advertisements to transmit responses back to the broadcaster. PAwR makes periodic advertising a bidirectional communication mode and allows large, one-to-many topologies to be created. Hence, in accordance with protocols that support periodic advertising with response, the embodiments of FIGS. 6 and 7 need not establish a communication session nor send/receive a request to initially send data (e.g. the operations at 601-602 and 701-702 may be omitted.

The foregoing examples of FIGS. 6 and 7 are implemented in connection with protocols that offer response acknowledgement to indicate whether a received data packet is good or bad. Additionally or alternatively, embodiments may be implemented with protocols that do not utilize (or can turn off) response acknowledgement. In some instances, it may be desirable to shift between higher and lower data rates, where the likelihood of errors is lower with the lower data rate due to various aspects of the lower data rate. Embodiments herein, successively transmitted the same data packet more than once in a bundle or set, based in part on the premise that at least one transmission of the data packet will arrive error-free. Embodiments in which the same data packet is successively transmitted more than once in a bundle or set may be a stand-alone implementation or provided in connection with a system that might otherwise shift between higher and lower data rates based upon environmental conditions or the importance with which certain data be received.

Additionally or alternatively, in certain conditions, it may be desirable to include repeated data packets (e.g., up to 5 total), such as for critical transmissions. The methods described herein can still be applied, even when repeating data packets (regardless of errors) to increase the successful rate of a single transmission.

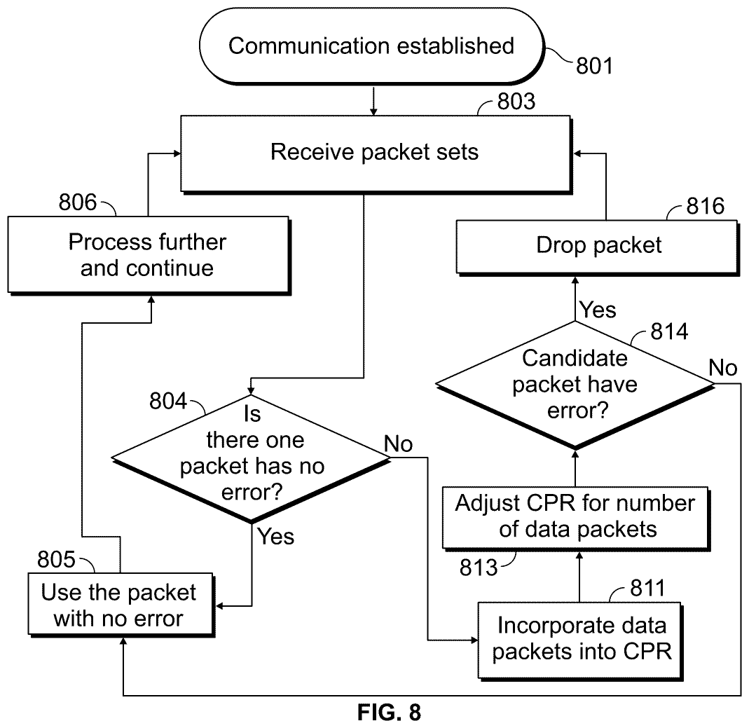

FIG. 8 illustrates a method for managing communication between at least first and second devices in accordance with embodiments herein utilizing a communication protocol that does not necessarily need to utilize response acknowledgments to inform the source/transmitting device when bad data packets are received. For example, FIG. 8 may be implemented in connection with protocols other than Bluetooth, such as through inductive telemetry. Although it is recognized that the embodiment of FIG. 8 is not limited to use with the inductive telemetry.

In the example of FIG. 8, the communication may be between an external device and IMD (e.g., an implantable cardioverter defibrillator) that supports first and second telemetry modes. The first and second telemetry modes may have different operating parameters such as first and second data rates, respectively. For example, the first telemetry mode may utilize inductive telemetry that transmits at a first data rate (e.g., 64-k), while the second telemetry mode may utilize inductive telemetry at a second data rate (e.g., 8K). Optionally, the first and second telemetry modes may utilize a common data rate with one of the telemetry modes retransmitting all data packets. For example, the first telemetry mode may transmit original data packets successively (at a first data rate) and only repeat a data packet as a “retry” data packet when the received data packet exhibits an error. In the first telemetry mode, the same data packet will only be repeated if it exhibits an error the first time received. The second telemetry mode may repeat the same data packet at least a predetermined number of times regardless of whether the received data packet exhibits an error. For example, the second telemetry mode may transmit every data packet a duplicate number of times such as 3 times, 5 times, etc. In the foregoing example, 3 or 5 data packets may be transmitted successively with duplicate/same data payload blocks and duplicate/same error detection blocks. As a further example, one data packet may be constructed with multiple data payload blocks within the same data packet (e.g., 3 or 5 data payload blocks). The one data packet may include 3 or 5 duplicate payload blocks and one error detection block).

In certain conventional approaches without the present improvements, in noise scenarios and/or while performing certain “high priority” operations (e.g., high voltage capacitor charging), the system may switch from a higher data rate (64-k telemetry) to a lower data rate (e.g., 8-k telemetry) to improve the communication quality. However, in accordance with the improvements herein for computers and communication technology, the system need not switch to a lower data rate and instead may continue to maintain the higher data rate due to the improved through-put afforded by functionally combining “bad” data packets until producing a resultant data packet that is a corrected version of the original data packet. Among other improvements, embodiments herein reduce a number of retry data packets that are transmitted which among other things conserves power and extends battery life.

Additionally or alternatively, the embodiment of FIG. 8 may be implemented, not with different data rates, but instead with devices that switch between 1) repeated data packets (e.g., a fixed number of times without regard for errors) and 2) transmitting each data packet once and only retrying when the original data packet exhibits an error.

The operations of FIG. 8 somewhat parallel certain operations of FIG. 6, but with other operations removed. The operations of FIG. 8 may be implemented by any or all of the various external, wearable and implantable devices described herein. The operations of FIG. 8 may be implemented in whole or in part within communication circuitry. Optionally some or all of the operations of FIG. 8 may be implemented by one or more processors, such as one or more processors within the communication circuitry and/or one or more processors more generally within the corresponding device (and not within the communication circuitry).

At 801, the receiving device establishes a communications link with a transmitting device. Optionally, the operations at 801 may be omitted entirely as the receiving device need not necessarily establish a communications link directly with a transmitting device in order to perform the remaining operations of FIG. 8. For example, the receiving device may represent an “observer” type of device that listens over a broadcasting channel for certain information. A broadcasting device may transmit over the broadcasting channel, a set of the same data packet (e.g, multiple data packets that contain the same payload block and same error detection block). The receiving observer device may collect the set of the same data packet and implement the remaining operations of FIG. 8.

At 803, a transceiver of the receiving device receives a set of two or more data packets that were duplicates of one another when transmitted. In other words, the transmitting device transmits a series of 2 or more identical data packets having the same payload block and same error detection code (with potentially different header information). The set of data packets are intended to be duplicative under the premise that at least one of the data packets will be received error free, thereby avoiding a need for acknowledgments or any other bidirectional information to inform the transmitting device whether the incoming data packets were error-free. Within an individual set, each of the data packets include the same payload block and same error detection block. In the present example, the first data packet within the set may be considered to represent the “original” data packet, while the rest of the data packets in the set may be considered to represent “retry” data packets.

At 804, the receiving device determines whether any one of the data packets within the set is error-free. To do so, the receiving device applies the same error detection process used to generate the error detection code in the data packets. The receiving device calculates corresponding error detection values associated with the content of each of the payload blocks for the data packets in the set and compares the corresponding error detection values to the corresponding error detection blocks to determine whether one or more of the data packets is error-free. When any one of the data packets is determined to be error-free, flow moves to 805 where the error-free data packet is utilized for further processing at 806.

Alternatively, when all of the data packets in the set are determined to exhibit an error, flow moves to 811. The operations at 811-814 generally correspond to the operations at 611, 613 and 614 in FIG. 6, except that all of the received data packets are incorporated into the candidate packet registered without regard for retries, and without initializing the CPR. At 811, the receiving device incorporates the data packets into the CPR utilizing one or more of the functional combinations discussed herein. At 813, the receiving device adjusts the CPR, cell by cell, based on the number of data packets included. For example, when the functional combination includes adding the data packets together bit by bit, the adjustment at 813 effectively calculates the average for each bit.

At 814, the receiving device determines whether the candidate packet within CPR still exhibits an error. The determination at 814 utilizes the same process as discussed above in connection with FIGS. 6 and 7. For example, the receiving device calculates an error detection value based on the content of the payload portion of CPR. The receiving device then compares the error detection value to the error detection portion within the CPR. When they do not match, the receiving device interprets this mismatch as an indication that the CPR still exhibits an error. Consequently, flow moves from 814 to 816 and the set of data packets is dropped. Thereafter, flow continues back to 803 and the next set of data packets is received and processed.

Returning to 814, alternatively, when the error detection value, calculated based on the payload portion of the CPR, matches the error detection block within CPR, the receiving device declares the candidate packet to have been corrected. Thus, flow moves to 805 where the payload content of the CPR is output as a resultant data packet that is declared/deemed to be a corrected version of the payload block originally transmitted from the transmitting device.

The process of FIG. 8 represents a somewhat streamlined implementation of the improvements herein that avoids the added decisions related to whether individual data packets represent retry packets and avoids the incremental initialization and updating to the CPR.

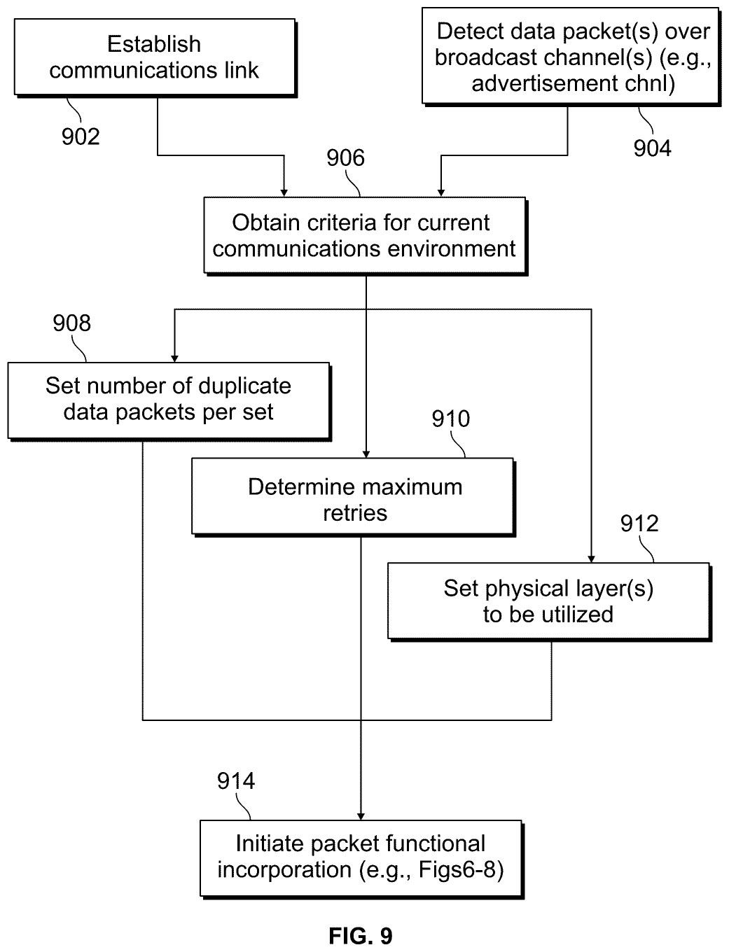

FIG. 9 illustrates a method implemented in accordance with embodiments herein. The operations at 902-912 may be implemented in whole or in part in combination with the operations of FIGS. 6-8. For example, the operations at 904-912 may be implemented between the operations at 601 and 602 (FIG. 6), between the operations at 701 and 702 (FIG. 7) and the operations at 801-803 (FIG. 8).

At 902, a communications link is established between 2 or more devices in accordance with the various examples described herein. Additionally or alternatively, at 904, a communications link need not be established. Instead, at 904, a receiving device detects one or more data packets over a broadcast channel or channels. For example, the operation 904 may correspond to an individual device listening over one or more advertising channels in accordance with any prior version of the BLE protocol or other protocols that support an advertise-response structure. As another example, at 904, multiple “observer” devices may be listening to a common broadcasting device, such as in accordance with BLE version 5.4 (e.g., the Periodic Advertisement with Response feature).

At 906, the device obtains one or more criteria associated with the current communications environment. Examples of the criteria may include noise, signal to noise ratio, number of drops packets previous experience, error rates and the like. As another example, the content received at 902 or 904 may include the criteria, such as when a transmitting or broadcasting device includes, in a data packet, information regarding the communications environment.

The operations at 908-912 may be implemented individually or collectively, with one or more of the operations at 908-912 entirely omitted. At 908, a number of duplicate data payloads is determined to be included in each set of data packets transmitted in accordance with the process of FIG. 8. For example, the process of FIG. 9 may determine that the communications environment is only slightly “harsh” and thus only warrant transmitting 3-4 duplicate data payloads in a single set of data packets (or in one data packet). Alternatively, the process may determine that the communication environment is very “harsh” and thus warrant transmitting up to 8 duplicate data payloads in a single set of data packets or single data packet.

At 910, the process may determine a maximum number of data packets to be retransmitted, such as in connection with the embodiments of FIGS. 6 and 7 that support acknowledgments/responses. For example, the process of FIG. 9 may determine that the communications environment is only slightly “harsh” and thus only warrant transmitting 3-4 retry data packets. Alternatively, the process may determine that the communication environment is very “harsh” and thus warrant transmitting 8 retry data packets.

The operation at 912 may be implemented in connection with devices that support more than one physical layer. For example, when the devices support the LE 1M and LE 2M physical layers, at 912, the process may determine whether the communications environment warrants the use of one or the other physical layer.

Following the determinations at 908-912, flow moves to 914 where the packet functional incorporation is initiated in accordance with the processes of FIGS. 6-8 and as described elsewhere herein.

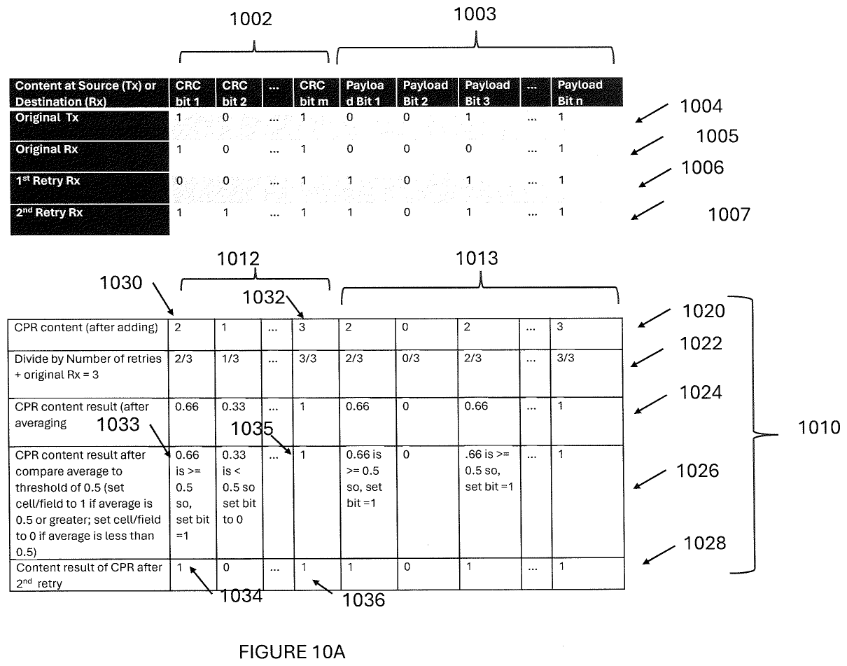

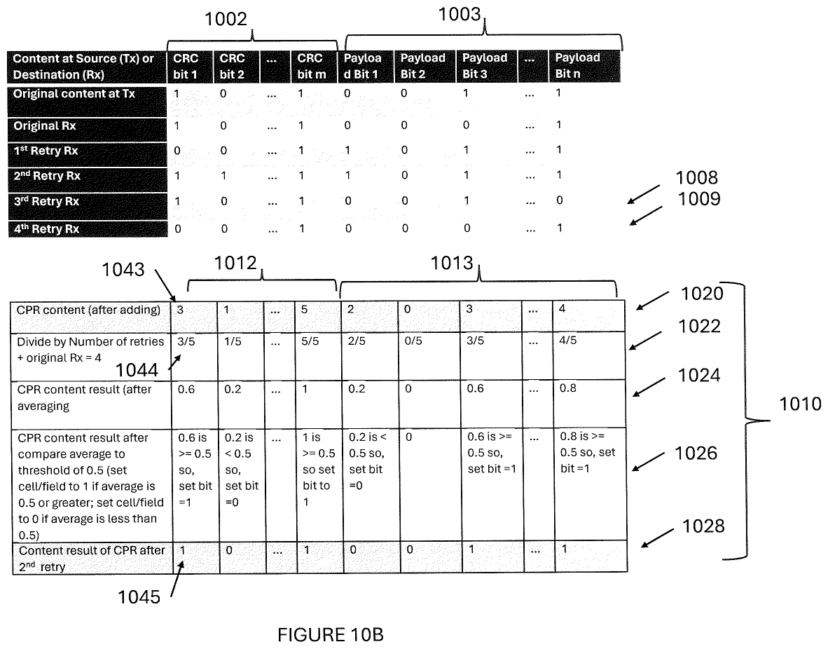

FIG. 10A illustrates example content of the candidate packet register in connection with a received original data packet, and first and second retry data packets in accordance with embodiments herein. In FIG. 10A, columns 1002 and 1003 correspond to the content (bit by bit) of an error detection block and content (bit by bit) of a payload block. Row 1004 corresponds to the content of the data packet as transmitted (during the original transmission and during each retry transmission). Row 1005 corresponds to the content of the data packet as originally received. Rows 1006 and 1007 correspond to the content of the data packet as received during first and second retries, respectively. The bits in the error detection portion of the original received (Rx) data packet have a content of 1, 0, . . . 1, while the bits in the error detection portion of the first retry data packet have a content of 0, 0, . . . , 1. It is understood that the portion denoted by “ . . . ” indicated an indefinite number of additional bits in the data packets.

FIG. 10A further illustrates example content of the cells in CPR 1010 at various points during processing. The columns 1012 and 1013 correspond to the error detection portion and payload portion of the CPR, respectively. Each row corresponds to a different point in the process of FIGS. 6 and 7. Row 1020 indicates the content (cell by cell) of the CPR after receiving the original, first and second retry data packets and incorporating all three into the CPR. Row 1020 is an example of the content of the CPR after the operation at 611 in FIG. 6 (and 711 in FIG. 7) at the time of the second retry. The cells in the error detection portion of the CPR have a content of 2, 1, . . . 3, where the portion denoted by “ . . . ” indicated an indefinite number of additional cells. Cell 1030 has the content of “2” as the sum of the first bit the original received data packet (1), the first bit in the first retry data packet (0), and the first bit in the second data packet (1).

Rows 1022-1026 correspond to the operation at 613 in FIGS. 6 and 713 in FIG. 7. Row 1022 indicates the operation of dividing the content of each cell in the CRP by the number of original and retries (e.g., in the present example, 3). Row 1024 indicates the resultant average value in each cell after the division at row 1022. Row 1026 illustrates the logical operation performed to round the average values in row 1024 to a logical “1” or “0”. For example, cell 1033 illustrates the operation of comparing the average (0.66) to the threshold (0.5). The average 0.66 is greater than or equal to 0.5 and thus the process sets the first cell of the CPR to a logical value of “1”, as noted at cell 1034. The averaging operation and rounding operation are performed at 613 in FIGS. 6 and 713 in FIG. 7.

As another example, cell 1032 has the content of “3” as the sum of the last CRC bit the original received data packet (1), the last CRC bit in the first retry data packet (1), and the last CRC bit in the second data packet (1). Cell 1035 illustrates the operation of comparing the average (1) to the threshold (0.5). The average 1 is greater than or equal to 0.5 and thus the process sets the first cell of the CPR to a logical value of “1”, as noted at cell 1036.

Row 1028 corresponds to the content of the CPR that is analyzed at 614 in FIGS. 6 and 714 in FIG. 7 to determine whether the CPR still includes an error. To perform the analysis, the payload portion of the CPR (the portion of row 1028 within columns 1013) is analyzed to determine a CPR error detection value (e.g., using a CRC calculation or check sum calculation). The CPR error detection value is calculated from the payload portion of the CPR using the same error detection algorithm as used at the transmitting device to create the error detection block in the original data packet as transmitted. The CPR error detection value is then compared to the content of the error detection portion of the CPR (the portion of row 1028 within columns 1012), corresponding to the decision at 614 and 714. In the example of FIG. 10A, the CPR error detection value would not match the content of the error detection portion of the CPR. Thus, the processes of FIGS. 6 and 7 would perform another iteration to obtain third and fourth retry data packets, as discussed further in connection with FIG. 10B.

FIG. 10B illustrates example content of the CPR following receipt of third and fourth retry data packets continuing the example of FIG. 10A. FIG. 10B corresponds to the same example for managing content of the candidate packet register in connection with the original receive and first and second retries of FIG. 10A, followed by third and fourth retries, in accordance with an embodiment herein. Elements of FIG. 10B that are the same as in FIG. 10A are given the same reference numbers, although it is understood that values within the CPR 1010 will be different from the same cells in the CPR 1010 in FIG. 10A given that third and fourth retry data packets have been added to the CPR 1010. The third retry data packet 1008 was received as 1,0, . . . , 1,0,0,1, . . . , 0. The fourth retry data packet 1009 was received as 0,0, . . . , 1,0,0,0, . . . , 1.

The values (e.g., value 1043) within the cells of row 1020 in the CPR have been updated by adding the bit values from the third and fourth retry data packets 1008 and 1009, now equaling 3,1, . . . , 5, 2, 0, 3, . . . , 4. At row 1022, the sums within row 1020 are divided by the number of data packets received (original and four retries). Cell 1044 shows the sum of “3” divided by “5”, the number of received data packets. Rows 1024 to 1028 have been updated similarly.

The row 1028 corresponds to the content of the CPR that is analyzed at 614 in FIGS. 6 and 714 in FIG. 7, after the fourth retry, to determine whether the CPR still includes an error. To perform the analysis, the payload portion of the CPR (the portion of row 1028 within columns 1013) is analyzed to determine a CPR error detection value (e.g., using a CRC calculation or check sum calculation). The CPR error detection value is calculated from the payload portion of the CPR using the same error detection algorithm as used at the transmitting device to create the error detection block in the original data packet as transmitted. The CPR error detection value is then compared to the content of the error detection portion of the CPR (the portion of row 1028 within columns 1012), corresponding to the decision at 614 and 714. In the example of FIG. 10A, the CPR error detection value would match the content of the error detection portion of the CPR. Thus, the process would declare the content of the CPR to be error free and a corrected version. Based on the analyzing, the process designates the content of the CPR to be a resultant packet and outputs the resultant packet as a corrected version of the data packet for further processing, storage and the like.

### Definitions

The term “original”, when used to describe a data packet shall mean a data packet that includes a payload that is being transmitted for a first time from one device to another device.

The terms “retry” and “retransmitted”, when used to describe a data packet, shall mean a data packet that includes a payload that has already been transmitted at least once from a first device to a second device and is now being resent at least a second time from the one device to the other device.

The “error check block” and an associated “payload block” may occur in a common data packet, be distributed between a series of two or more packets, and/or located in separate and distinct packets. For example, a data packet may include a payload portion preceded or followed by an error check portion in the same data packet (e.g., a check sum field, CRC field, etc.). In the foregoing example, the payload portion and error check portion are in the same data packet. Additionally or alternatively, a first packet may contain a payload portion and a second packet may contain the error check portion, thereby affording an example with the error check portion and the payload portion in different, but associated packets. Additionally or alternatively, the payload portion may be distributed between a series of two or more packets, while the error check portion is at least one of i) included in one of the series of two or more packets, ii) included in a packet separate from the series of two or more packets, or iii) distributed between each of the two or more packets.

The term “error”, when used to describe a packet, shall mean that the packet contains a data block that does not match a corresponding source data block transmitted from a source device. More specifically, in connection with data packet transmission and retransmission from a source device to a destination device, a received data packet, at a destination device, shall exhibit an error when the received data packet contains a data block that differs from the data block transmitted or retransmitted from the source device. Throughout the discussion, when the terms “an error”, “the error”, “errors” and “one or more errors” are used to describe original and retry data packets, it is understood that the original data packet and the corresponding retry data packet are considered to exhibit some type of error, but not necessarily the same specific erroneous data bits and/or same specific mis-match of an error check segment (e.g., CRC code) and a data segment (e.g., payload).

The terms “bad”, “error” and “errors”, when used to describe a condition of a data packet received at a destination/receiving device or candidate packet register, shall mean that an integrity of a content of a payload block of the data packet or candidate packet register cannot be verified by an error detection process applied to the data packet or candidate packet register. As one example, when the data packet includes an error detection block, in addition to a payload block, the data packet will be deemed to exhibit an error when an error detection process calculates an error detection value associated with the content of the payload block and determines that the error detection value does not match a content of the error detection block. Similarly, the candidate packet register will be deemed to exhibit an error when an error detection process calculates an error detection value associated with the content of the payload block of the register and determines that the error detection value does not match a content of the error detection block of the register.

Further, when the terms “error” and “errors”, are used to describe an original data packet and one or more corresponding retry data packets, it is understood that the originally received data packet and the one or more corresponding received retry data packets do not necessarily include the same specific error(s) in the same bit location(s). Instead, when an original data packet and one or more corresponding retry data packets are deemed to exhibit “an error”, “the error”, “errors”, the original and retry data packets may exhibit one or more errors in the same or different bit locations.

The terms “good”, “correct” and “error-free”, when used to describe a condition of a data packet received at a destination/receiving device or candidate packet register, shall mean that an integrity of a content of a payload block of the data packet or candidate packet register has been verified by an error detection process applied to the data packet or candidate packet register. As an example, the data packet or candidate packet register will be deemed to be error free or a corrected version of a transmitted data packet, when an error detection process calculates an error detection value associated with the content of the payload block and determines that the error detection value does match a content of the error detection block.

The term “corrected version”, when used to describe a condition of a resultant packet output from the candidate packet register, shall mean that a content of a payload block of the resultant packet has been verified by an error detection process and is deemed to correspond to a content of a payload block of an original data packet as transmitted from a source device.

The terms “behavior related medical data” and “BRM data” shall mean information indicative of an action or conduct by a patient that will affect one or more physiologic characteristics of interest and/or information indicative of a present state experienced by a patient in connection with a physiologic characteristic of interest. As nonlimiting examples of information indicative of an act or conduct, BRM data may represent information related to a patient's diet (e.g., what, when and how much a patient ate or drank), information related to whether a patient is following a physician's instructions (e.g., exercising, walking, following a fluid regiment, taking medication at prescribed times), information related to nutritional supplements (e.g., what, when and how much a patient is taking as nutritional supplements), self-reported quality of life information from the patient, signs and symptoms indicating fatigue, lack of mobility/exercise and the like. The foregoing examples concern BRM data that is directly relate to actions and/or conduct by the patient. Optionally, the BRM data may indirectly relate to actions and/or conduct by the patient. For example, the BRM data may indicate how often and/or volumes of certain food products and liquids ordered by the patient through a home delivery service (e.g., how often and in what volume the patient orders certain groceries and other food products that may be delivered to a patient's home).

Further, as nonlimiting examples of information indicative of a present state, the BRM data may represent information indicating how a patient feels (e.g., headaches, shortness of breath, tired, chest pains). The BRM data may be manually entered by the patient or a third-party through various types of PDE devices. Optionally, the BRM data may be automatically entered by a PDE device based on electronic monitoring of actions and conduct by the patient, as well as other types of sensors.

The term “BGA test device” shall mean any and all equipment, devices, disposable products utilized to collect and analyze a BGA. The BGA test device may implement one or more of the methods, devices and systems described in the following publications, all of which are incorporated herein by reference in their entireties: U.S. Pat. No. 8,514,086, entitled “DISPLAYS FOR A MEDICAL DEVICE”, issued Aug. 20, 2013; U.S. Patent Publication Number 2011/0256024, entitled “MODULAR ANALYTE MONITORING DEVICE”, published Oct. 20, 2011; U.S. Patent Publication Number 2010/0198142, entitled “MULTIFUNCTION ANALYTE TEST DEVICE AND METHODS THEREFORE”, published Aug. 5, 2010; U.S. Patent Publication Number 2011/0160544, entitled “SYSTEM AND METHOD FOR ANALYSIS OF MEDICAL DATA TO ENCOURAGE HEALTHCARE MANAGEMENT”, published Jun. 30, 2011; U.S. Pat. No. 5,294,404, entitled “REAGENT PACK FOR IMMUNOASSAYS” issued Mar. 15, 1994; U.S. Pat. No. 5,063,081, entitled “METHOD OF MANUFACTURING A PLURALITY OF UNIFORM MICROFABRICATED SENSING DEVICES HAVING AN IMMOBILIZED LIGAND RECEPTOR” issued Nov. 5, 1991; U.S. Pat. No. 7,419,821, entitled “APPARATUS AND METHODS FOR ANALYTE MEASUREMENT AND IMMUNOASSAY” issued Sep. 2, 2008; U.S. Patent Publication Number 2004/0018577, entitled “MULTIPLE HYBRID IMMUNOASSAYS” published Jan. 29, 2004; U.S. Pat. No. 7,682,833, entitled “IMMUNOASSAY DEVICE WITH IMPROVED SAMPLE CLOSURE” issued Mar. 23, 2010; and U.S. Pat. No. 7,723,099, entitled “IMMUNOASSAY DEVICE WITH IMMUNO-REFERENCE ELECTRODE” issued May 25, 2010, all of which are expressly incorporated by reference in their entireties.

The term “body generated analyte” shall mean a test substance or specimen that is naturally generated by or naturally present in a human body. The test substance or specimen may be in liquid form (e.g., blood or other bodily fluid), solid form (e.g., tissue, fat, muscle, bone, or other organ-based material), gas form, cellular form or otherwise. Non-limiting examples of body generated analytes include hematocrit, troponin, CKMB, BNP, beta human chorionic gonadotropin (bHCG), carbon dioxide partial pressure (pCO.sub.2), partial pressure oxygen (pO.sub.2), pH, PT, ACT, activated partial thromboplastin time (APTT), sodium, potassium, chloride, calcium, urea, glucose, creatinine, lactate, oxygen, and carbon dioxide, thyroid stimulating hormone, parathyroid hormone, D-dimer, prostate specific antibody, TCO₂, Anion Gap, ionized calcium, urea nitrogen, lactose, hemoglobin, pH, PCO₂, PO₂, HCO₃, Base Excess, O₂, ACT Kaolin, ACT Celite, PT/INR, β-hCG, cTnl, CK-MB, BNP and the like, and combinations thereof. The analyte may be tested in a liquid sample that is whole blood, however other samples can be used including blood, serum, plasma, urine, cerebrospinal fluid, saliva and amended forms thereof. Amendments can include diluents and reagents such as anticoagulants and the like.

The terms “cardiac activity signal”, “cardiac activity signals”, “CA signal” and “CA signals” (collectively “CA signals”) are used interchangeably throughout to refer to measured signals indicative of cardiac activity by a region or chamber of interest. For example, the CA signals may be indicative of impedance, electrical or mechanical activity by one or more chambers (e.g, left or right ventricle, left or right atrium) of the heart and/or by a local region within the heart (e.g., impedance, electrical or mechanical activity at the AV node, along the septal wall, within the left or right bundle branch, within the purkinje fibers). The cardiac activity may be normal/healthy or abnormal/arrhythmic. An example of CA signals includes EGM signals. Electrical based CA signals refer to an analog or digital electrical signal recorded by two or more electrodes, where the electrical signals are indicative of cardiac activity. Heart sound (HS) based CA signals refer to signals output by a heart sound sensor such as an accelerometer, where the HS based CA signals are indicative of one or more of the S1, S2, S3 and/or S4 heart sounds. Impedance based CA signals refer to impedance measurements recorded along an impedance vector between two or more electrodes, where the impedance measurements are indicative of cardiac activity.

The term “IMD data” shall refer to any and all types of information and signals conveyed from an implantable medical device to a local or remote external device. Nonlimiting examples of IMD data include cardiac activity signals (e.g., intracardiac electrogram or IEGM signals), impedance signals (e.g., cardiac, pulmonary or transthoracic impedances), accelerometer signatures (e.g., activity signals, posture/orientation signals, heart sounds), pulmonary arterial pressure signals, MCS rpm levels, MCS flow rates, device alerts and the like.

The term “obtains” and “obtaining”, as used in connection with data, signals, information and the like, include at least one of i) accessing memory of an external device or remote server where the data, signals, information, etc. are stored, ii) receiving the data, signals, information, etc. over a wireless communications link between the IMD and a local external device, and/or iii) receiving the data, signals, information, etc. at a remote server over a network connection. The obtaining operation, when from the perspective of an IMD, may include sensing new signals in real time, and/or accessing memory to read stored data, signals, information, etc. from memory within the IMD. The obtaining operation, when from the perspective of a local external device, includes receiving the data, signals, information, etc. at a transceiver of the local external device where the data, signals, information, etc. are transmitted from an IMD and/or a remote server. The obtaining operation may be from the perspective of a remote server, such as when receiving the data, signals, information, etc. at a network interface from a local external device and/or directly from an IMD. The remote server may also obtain the data, signals, information, etc. from local memory and/or from other memory, such as within a cloud storage environment and/or from the memory of a workstation or clinician external programmer.

The terms “processor,” “a processor”, “one or more processors” and “the processor” shall mean one or more processors. The one or more processors may be implemented by one, or by a combination of more than one implantable medical device, a wearable device, a local device, a remote device, a server computing device, a network of server computing devices and the like. The one or more processors may be implemented at a common location or at distributed locations. The one or more processors may implement the various operations described herein in a serial or parallel manner, in a shared-resource configuration and the like.

The term “real-time” refers to a time frame contemporaneous with normal or abnormal episode occurrences. For example, a real-time process or operation would occur during or immediately after (e.g., within minutes or seconds after) a cardiac event, a series of cardiac events, an arrhythmia episode, and the like. For example, the term “real-time” may refer to a time period substantially contemporaneous with an event of interest. The term “real-time,” when used in connection with collecting and/or processing data utilizing an IMD, shall refer to processing operations performed substantially contemporaneous with a physiologic event of interest experienced by a patient. By way of example, in accordance with embodiments herein, cardiac activity signals are analyzed in real time (e.g., during a cardiac event or within a few minutes after the cardiac event). The term “real-time,” when used in connection with a body generated analyte, shall refer to operations performed substantially contemporaneous with an occurrence of a characteristic of interest in a malnutrition state experienced by the patient. By way of example, in accordance with embodiments herein, the body generated analyte may correspond to serum albumin that is analyzed and utilized in a diagnosis and treatment recommendation. The analysis of the serum albumin and generation of the diagnosis and treatment recommendation are performed in real-time, namely while the patient is experiencing a certain malnutrition state, not to exceed 24 hours from the time the BGA was collected.

The foregoing embodiments are described primarily in connection with BLE and inductive telemetry protocols. For example, embodiments may be implemented in connection with various Bluetooth protocols as defined in the following documents: Bluetooth Core Specification, Version 4.0, version date Jun. 30, 2010; Bluetooth Core Specification, Version 4.2, version date Dec. 2, 2014; Bluetooth Core Specification, Version 5.0, version date Dec. 6, 2016; and Bluetooth Core Specification, Version 5.4, version date Jan. 31, 2023, all of which are expressly incorporated by reference in their entireties.

The foregoing embodiments are described primarily in connection with implantable and wearable medical devices. For example, embodiments may be implemented in connection with one or more of the sensors, implantable devices, wearable patches, drug delivery pumps and external devices described in the following documents: U.S. application Ser. No. 16/930,791, filed Jul. 16, 2020 and titled “METHODS, DEVICES AND SYSTEMS FOR HOLISTIC INTEGRATED HEALTHCARE PATIENT MANAGEMENT” (Attorney Docket 13-0356US1) (Client Docket 13564USO1); U.S. Pat. No. 9,216,285 “Leadless Implantable Medical Device Having Removable And Fixed Components” and U.S. Pat. No. 8,831,747 “Leadless Neurostimulation Device And Method Including The Same”; U.S. Pat. No. 10,765,860, titled “Subcutaneous Implantation Medical Device With Multiple Parasternal-Anterior Electrodes”; U.S. Pat. No. 10,722,704, titled “Implantable Medical Systems And Methods Including Pulse Generators And Leads”; U.S. Pat. No. 11,045,643, titled “Single Site Implantation Methods For Medical Devices Having Multiple Leads”; U.S. Pat. No. 9,265,428 entitled “Implantable Wireless Sensor”, U.S. Pat. No. 8,278,941 entitled “Strain Monitoring System and Apparatus”, U.S. Pat. No. 8,026,729 entitled “System and Apparatus for In-Vivo Assessment of Relative Position of an Implant”, U.S. Pat. No. 8,870,787 entitled “Ventricular Shunt System and Method”, and U.S. Pat. No. 9,653,926 entitled “Physical Property Sensor with Active Electronic Circuit and Wireless Power and Data Transmission”; U.S. Pat. No. 10,729,346, titled “METHOD AND SYSTEM FOR SECOND PASS CONFIRMATION OF DETECTED CARDIAC ARRHYTHMIC PATTERNS”; U.S. Pat. No. 11,020,036, titled “METHOD AND SYSTEM TO DETECT R-WAVES IN CARDIAC ARRHYTHMIC PATTERNS”; U.S. Pat. No. 10,874,322, titled “METHOD AND SYSTEM TO DETECT POST VENTRICULAR CONTRACTIONS IN CARDIAC ARRHYTHMIC PATTERNS”; and U.S. Pat. No. 10,777,880, titled “METHOD AND SYSTEM TO DETECT NOISE IN CARDIAC ARRHYTHMIC PATTERNS”; U.S. application Ser. No. 17/192,961, filed Mar. 5, 2021, (attorney docket 13-0397US1) (client docket 13967USO1), titled “SYSTEM FOR VERIFYING A PATHOLOGIC EPISODE USING AN ACCELEROMETER”; U.S. application Ser. No. 16/869,733, filed May 8, 2020, (attorney docket 13-0396US1) (client docket 13964USO1), titled “METHOD AND DEVICE FOR DETECTING RESPIRATION ANOMALY FROM LOW FREQUENCY COMPONENT OF ELECTRICAL CARDIAC ACTIVITY SIGNALS;” U.S. application Ser. No. 17/194,354, filed Mar. 8, 2021, (Attorney docket 13-0395US1) (client docket 13949USO1), titled “METHOD AND SYSTEMS FOR HEART CONDITION DETECTION USING AN ACCELEROMETER”; US20240023024A1, titled “SYSTEMS, DEVICES AND METHODS FOR POWER-EFFICIENT WIRELESS COMMUNICATIONS BETWEEN ELECTRONIC DEVICES”, files 2023 Aug. 25; US20230404441A1, titled “SYSTEMS, DEVICES, AND METHODS FOR MEAL-RELATED ANALYTE RESPONSE MONITORING”, filed 2023 Apr. 25; US20220150308A1, titled “TRANSMITTING ANALYTE DATA USING LOW-POWER INSTRUCTION SETS,” filed 2022 Jan. 21; US20220369926A1, titled “SYSTEMS, DEVICES, AND METHODS FOR SENSOR COMMUNICATIONS,” filed 2022 Apr. 26; US20220167885A1, titled “SYSTEMS, DEVICES, AND METHODS FOR ESTABLISHING AND/OR MAINTAINING SYNCHRONIZATION BETWEEN ENTITIES IN AN ANALYTE MONITORING ENVIRONMENT,” filed 2021 Jul. 1; US20230337976A1, titled “SYSTEMS, DEVICES, AND METHODS FOR WELLNESS AND NUTRITION MONITORING AND MANAGEMENT USING ANALYTE DATA,” filed 2023 Jun. 22; US20240053324A1, titled “SYSTEMS, DEVICES, AND METHODS FOR WIRELESS COMMUNICATIONS IN ANALYTE MONITORING SYSTEMS,” filed 2023 Sep. 6, all of which are expressly incorporated by reference in their entireties.

While the foregoing embodiments have been described in connection with communication with a medical device, the present invention is not limited to medical systems and methods. Instead, the systems and methods described herein may be implemented in connection with communication between two or more non-medical devices. For example, embodiments may be implemented in connection with any two or more devices that utilize a wireless communication protocol that includes error detection, such as Bluetooth low energy, Bluetooth, Medical Implant Communication Service (MICS), ZigBee, Wi-Fi, ultrawideband, inductive telemetry, near field communications (NFC) and the like (e.g., between laptop computers, smartTVs, tablet devices, smart phones, routers, wireless security devices, wireless home management devices and the like).

### Closing

It should be clearly understood that the various arrangements and processes broadly described and illustrated with respect to the Figures, and/or one or more individual components or elements of such arrangements and/or one or more process operations associated of such processes, can be employed independently from or together with one or more other components, elements and/or process operations described and illustrated herein. Accordingly, while various arrangements and processes are broadly contemplated, described and illustrated herein, it should be understood that they are provided merely in illustrative and non-restrictive fashion, and furthermore can be regarded as but mere examples of possible working environments in which one or more arrangements or processes may function or operate.

Some or all of the Figures herein illustrates various methods and processes implemented in accordance with embodiments herein. The operations herein may be implemented by hardware, firmware, circuitry and/or one or more processors housed partially an/or entirely within an IMD, a local external device, remote server or more generally within a healthcare system. Optionally, the operations herein may be partially implemented by an IMD and partially implemented by a local external device, remote server or more generally within a healthcare system. For example, the IMD includes IMD memory and one or more IMD processors, while each of the external devices/systems (ED) (e.g., local, remote or anywhere within the healthcare system) include ED memory and one or more ED processors.

As will be appreciated by one skilled in the art, various aspects may be embodied as a system, method or computer (device) program product. Accordingly, aspects may take the form of an entirely hardware embodiment or an embodiment including hardware and software that may all generally be referred to herein as a “circuit,” “module” or “system.” Furthermore, aspects may take the form of a computer (device) program product embodied in one or more computer (device) readable storage medium(s) having computer (device) readable program code embodied thereon.

Any combination of one or more non-signal computer (device) readable medium(s) may be utilized. The non-signal medium may be a storage medium. A storage medium may be, for example, an electronic, magnetic, optical, electromagnetic, infrared, or semiconductor system, apparatus, or device, or any suitable combination of the foregoing. More specific examples of a storage medium would include the following: a portable computer diskette, a hard disk, a random access memory (RAM), a dynamic random access memory (DRAM), a read-only memory (ROM), an erasable programmable read-only memory (EPROM or Flash memory), a portable compact disc read-only memory (CD-ROM), an optical storage device, a magnetic storage device, or any suitable combination of the foregoing.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of network, including a local area network (LAN) or a wide area network (WAN), or the connection may be made through other devices (for example, through the Internet using an Internet Service Provider) or through a hard wire connection, such as over a USB connection. For example, a server having a first processor, a network interface, and a storage device for storing code may store the program code for carrying out the operations and provide this code through its network interface via a network to a second device having a second processor for execution of the code on the second device.

Aspects are described herein with reference to the Figures, which illustrate example methods, devices and program products according to various example embodiments. The program instructions may be provided to a processor of a general-purpose computer, special purpose computer, or other programmable data processing device or information handling device to produce a machine, such that the instructions, which execute via a processor of the device implement the functions/acts specified. The program instructions may also be stored in a device readable medium that can direct a device to function in a particular manner, such that the instructions stored in the device readable medium produce an article of manufacture including instructions which implement the function/act specified. The program instructions may also be loaded onto a device to cause a series of operational steps to be performed on the device to produce a device implemented process such that the instructions which execute on the device provide processes for implementing the functions/acts specified.

The units/modules/applications herein may include any processor-based or microprocessor-based system including systems using microcontrollers, reduced instruction set computers (RISC), application specific integrated circuits (ASICs), field-programmable gate arrays (FPGAs), logic circuits, and any other circuit or processor capable of executing the functions described herein. Additionally, or alternatively, the modules/controllers herein may represent circuit modules that may be implemented as hardware with associated instructions (for example, software stored on a tangible and non-transitory computer readable storage medium, such as a computer hard drive, ROM, RAM, or the like) that perform the operations described herein. The above examples are exemplary only, and are thus not intended to limit in any way the definition and/or meaning of the term “controller.” The units/modules/applications herein may execute a set of instructions that are stored in one or more storage elements, in order to process data. The storage elements may also store data or other information as desired or needed. The storage element may be in the form of an information source or a physical memory element within the modules/controllers herein. The set of instructions may include various commands that instruct the modules/applications herein to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to user commands, or in response to results of previous processing, or in response to a request made by another processing machine.

It is to be understood that the subject matter described herein is not limited in its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It should be recognized that, to the extent embodiments herein are described to apply certain mathematical combinations of select variables, the same variables may be combined in other mathematical combinations that are also indicative of the same result. For example, when a single data point is utilized for a particular variable, additionally or alternatively, a mean, average, sum, or other mathematical combination of multiple data points may be utilized for the same variable.

It is to be understood that the above description is intended to be illustrative, and not restrictive. For example, the above-described embodiments (and/or aspects thereof) may be used in combination with each other. In addition, many modifications may be made to adapt a particular situation or material to the teachings herein without departing from its scope. While the dimensions, types of materials and coatings described herein are intended to define various parameters, they are by no means limiting and are illustrative in nature. Many other embodiments will be apparent to those of skill in the art upon reviewing the above description. The scope of the embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects or order of execution on their acts.

What is claimed is:

1. A method of communication between a first medical device and a second device utilizing a predetermined protocol, the method comprising:
utilizing at least one of communication circuitry or a processor, within one of the first medical device and the second device, for,
receiving a data packet;
determining whether the data packet exhibits an error;
when the data packet exhibits the error, incorporating the data packet into a candidate packet register (CPR) within the one of the first medical device and the second device;
analyzing content the CPR for errors; and
based on the analyzing, designating the content of the CPR to be a resultant packet and outputting the resultant packet as a corrected version of the data packet.

2. The method of claim 1, wherein the data packet includes a payload block and an error detection block, the incorporating operation comprising adding the payload and error detection blocks to corresponding payload and error detection blocks of the CPR.

3. The method of claim 2, further comprising rounding the payload and error detection blocks of the CPR on a bit-by-bit basis, based on a number of data packets added to the CPR.

4. The method of claim 2, wherein the analyzing operation includes calculating an error detection value associated with the content of the payload block of the CPR and determining when the error detection value matches the error detection block within the CPR.

5. The method of claim 4, wherein the receiving includes receiving an original data packet and a corresponding retry data packet, the incorporating operation including the original data packet in the CPR and adding the retry data packet to the CPR.

6. The method of claim 1, wherein the first medical device (MD) represents at least one of an implantable medical device, an analyte sensor or a drug delivery pump, and the second device represents an external device (ED), wherein the protocol supports transmission of retry data packets, the method comprising:
establishing a communications link between the first MD and the ED, the communications link having connection intervals in accordance with the protocol; and
wherein the CPR represents a dynamic average check (DAC) packet.

7. The method of claim 1, wherein the receiving, determining, incorporating and analyzing operations are performed a first iteration for an original data packet and are repeated, at least a second iteration, for a first retry data packet presenting an attempt to retransmit the original data packet.

8. The method of claim 7, wherein, when the first retry data packet is determined to exhibit the error, the method repeating the receiving and determining a third iteration in connection with a second retry data packet representing a second attempt to retransmit the new data packet.

9. The method of claim 1, wherein the determining whether the data packet exhibits an error applies and the analyzing the content the CPR for errors apply a common error detection algorithm to payload and error detection blocks in the data packet and to payload and error detection blocks in the CPR.

10. The method of claim 1, wherein the incorporating operation effectively removed one or more errors from the content of the CPR.

11. A system, comprising:
a first medical device and a second device configured to communicate with one another utilizing a predetermined protocol;
at least one of communication circuitry or a processor, within one of the first medical device and the second device, configured to:
receive a data packet;
determine whether the data packet exhibits an error;
when the data packet exhibits the error, incorporate the data packet into a candidate packet register (CPR) within the one of the first medical device and the second device;
analyze content the CPR for errors; and
based on the analysis, designate the content of the CPR to be a resultant packet and output the resultant packet as a corrected version of the data packet.

12. The system of claim 11, wherein the data packet includes a payload block and an error detection block, the incorporate operation adding the payload and error detection blocks to corresponding payload and error detection blocks of the CPR.

13. The system of claim 12, wherein the at least one of communication circuitry or processor configured to round the payload and error detection blocks of the CPR on a bit-by-bit basis, based on a number of data packets added to the CPR.

14. The system of claim 11, wherein the analyze operation includes calculating an error detection value associated with the content of the payload block of the CPR and determining when the error detection value matches the error detection block within the CPR.

15. The system of claim 11, wherein the first medical device (MD) represents at least one of an implantable medical device, an analyte sensor or a drug delivery pump, and the second device represents an external device (ED), wherein the protocol supports transmission of retry data packets, the first MD and the ED configured to establish a communications link having connection intervals in accordance with the protocol; and wherein the CPR represents a dynamic average check (DAC) packet.

16. The system of claim 11, wherein the receive, determine, incorporate and analyze operations are performed a first iteration for an original data packet and are repeated, at least a second iteration, for a first retry data packet presenting an attempt to retransmit the original data packet.

17. The system of claim 16, wherein, during the first iteration, the CPR is initialized with a payload block and an error detection block from the original data packet, and, during the second iteration, the CPR is updated by adding payload and error detection blocks from the first retry data packet to the payload and error detection blocks in the CPR.

18. The system of claim 11, wherein the receiving the data packet includes receiving a set of at least two data packets that, when transmitted, included a same payload block and a same error detection block.

19. The system of claim 11, wherein the receiving and determining are repeated in connection with a new data packet, followed by corresponding first and second retry data packets, the new, first retry and second retry data packets exhibiting the error, the incorporating including combining the new, first retry and second retry data packets to form the DAC packet, the DAC packet designated to be error free and output.

20. The system of claim 11, wherein, prior to the incorporating, the CPR already includes a first payload block and first error detection block, the incorporating operation including incorporating a second data packet into the CPR by applying at least one of a mathematical or logical combination to i) second payload and error detection blocks of the second data packet and ii) the first payload and error detection blocks, respectively.

21. A method of communication between a first device and a second device utilizing a predetermined protocol, the method comprising:
utilizing at least one of communication circuitry or a processor, within one of the first device and the second device, for,
receiving a data packet;
determining whether the data packet exhibits an error;
when the data packet exhibits the error, incorporating the data packet into a candidate packet register (CPR) within the one of the first device and the second device;
analyzing content the CPR for errors; and
based on the analyzing, designating the content of the CPR to be a resultant packet and output the resultant packet as a corrected version of the data packet.

22. The method of claim 21, wherein the data packet includes a payload block and an error detection block, the incorporating operation comprises adding the payload and error detection blocks to corresponding payload and error detection blocks of the CPR.

23. The method of claim 22, further comprising rounding the payload and error detection blocks of the CPR on a bit-by-bit basis, based on a number of data packets added to the CPR.
